(19) United States
(12) Patent Application Publication
(10) Pub. No.: US 2016/0279154 A1
(43) Pub. Date: Sep. 29, 2016

(54) MITOCONDRIALLY-TARGETED TIMOQUINONES AND TOLUQUINONES

(71) Applicant: MITOTECH SA, Luxembourg (LU)

(72) Inventors: VLADIMIR P SKULACHEV, MOSCOW (RU); MAXIM V SKULACHEV, MOSCOW (RU)

(21) Appl. No.: 15/059,921

(22) Filed: Mar. 3, 2016

Related U.S. Application Data
(63) Continuation of application No. PCT/IB2014/001658, filed on Apr. 11, 2014.

(51) Int. Cl. A61K 31/66; A61K 31/352

(52) U.S. Cl. CPC A61K 31/66; A61K 31/352

(57) ABSTRACT

The invention provides timoquinone-based mitochondrially-targeted antioxidants (MTAs) and methods for their use.

### BACKGROUND OF THE INVENTION

1. Field of the Invention

The invention relates to the fields of pharmacology and medicine, and in particular to mitochondrially-targeted quinones and quinoles and pharmaceutical compositions based on these compounds.

2. Summary of the Related Art

Promising therapeutic properties of mitochondrially-targeted antioxidants (MTAs) have been described (see, e.g., US2008176929; Skulachev et al (2009), Biochim. Biophys. Acta, 1787:437-61). At the moment several quinones are disclosed as the antioxidant moieties of mitochondrially targeted antioxidants, namely ubiquinone in MitoQ (U.S. Pat. No. 6,331,532), plastoquinone in SkQ1, SkQ3 SkQR1, SkQB1, SkQBP1 (WO2011059355) and methylplastoquinone in SkQ3 (WO2011059355).

Experiments with the above mentioned compounds demonstrated that the antioxidant activity of MTA depends on the structure of the quinone moiety (see for example FIG. 4b in Antonenko et al, (2008), Biochemistry (Moscow), 73, pp. 1273-1287).

As antioxidant moieties can provide different antioxidant properties, there is a need for new MTAs having new antioxidant moieties.

### BRIEF SUMMARY OF THE INVENTION

The present invention provides new MTAs comprising derivatives of timoquinone (see formula 1) as antioxidant moieties.

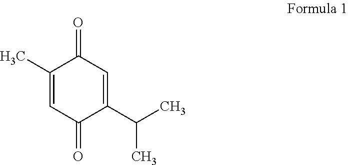

These timoquinone-based MTAs can be described by the general formula 2:

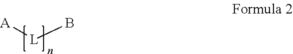

A is the timoquinone-derived antioxidant moiety of Formula 3:

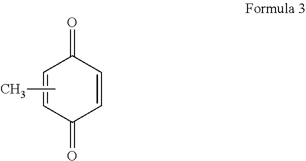

or the reduced form thereof, wherein the CH3 group is attached to any free position of the quinone ring;
or the timoquinone-derived antioxidant moiety of Formula 4:

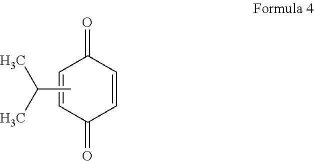

or the reduced form thereof; wherein the isopropyl group is attached to any free position of the quinone ring; or the timoquinone-derived antioxidant moiety of Formula 5:

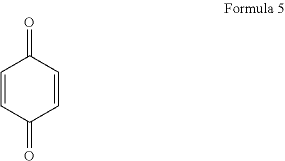

or the reduced form thereof.

L is a linker group, comprising: a) a straight or branched hydrocarbon chain optionally substituted by one or more double or triple bonds, or ether bond, or ester bond, or C—S, or S—S, or peptide bond; and which is optionally substituted by one or more substituents preferably selected from alkyl, alkoxy, halogen, keto group, amino group; or b) a natural isoprene chain;

n is an integer from 1 to 20; and

B is a targeting group comprising: a) a Skulachev-ion Sk (Sk⁺ Z⁻) wherein: Sk is a lipophilic cation or a lipophillic metalloporphyrin, and Z is a pharmaceutically acceptable anion; or b) an amphiphillic zwitterion.

### BRIEF DESCRIPTION OF THE DRAWINGS

FIG. 1A shows yield of chromatography of Qt-ClOBr; FIG. 1B shows results.

FIG. 2 shows results of HPLC and UV spectrum analysis of SkQT.

FIG. 3 shows results of HPLC of SkQT in isocratic mode.

FIGS. 4A and 4B show results of 1H NMR of SkQT at different temperatures.

FIG. 5 shows results of LC-MS of SkQRT1.

FIG. 6 shows results of HPLC of SkQRT1.

FIG. 7 shows results of LC-MS of SkQB.

FIG. 8 shows that SkQT protects mitochondria from oxidative damage.

FIG. 9 shows that SkQT can be reduced by mitochondria.

FIG. 10 shows that the inhibitor myxothiasole prevents further reduction of SkQT by mitochondria.

FIG. 11 shows that SkQT efficiently prevents peroxide-induced cell death.

FIG. 12 shows the dose-dependency of prevention of peroxide-induced cell death by SkQT.

FIG. 13 shows effect of various concentrations of SkQT (marked as SkQt on the figure) on H₂0₂ formation by Y. lipolytica mitochondria.

FIG. 14 shows effect of various concentrations of SkQ1 on H₂0₂ formation by Y. lipolytica mitochondria.

FIG. 15 shows comparison of effects of SkQ1 and SkQT on H₂0₂ formation by rat liver mitochondria.

FIG. 16 shows accumulation of SkQR1 and SkQRT1 in HeLa cells,

FIG. 17 shows increased accumulation of SkQR1 and SkQRT1 after inhibition of MDR proteins with Pluronic® L61. After 60 minutes, the medium was changed to control medium without SkQs (indicated with vertical line).

FIG. 18 shows the accumulation of SkQR1 and SkQRT1 in normal (non-tumor) cells and human fibroblasts. After 60 minutes, the medium was changed to control medium without SkQs (indicated with vertical line).

FIG. 19 shows antimycin-sensitive reduction of SkQB by energized mitochondria.

FIG. 20 shows that SkQB antioxidant activity is lower than that of SkQ1.

FIG. 21 shows that SkQB stimulates greater H2O2 production in energized mitochondria relative to SkQ1.

FIG. 22 shows that SkQB provides weaker protection from H₂O₂ killing of fibroblasts than SkQ1 and SkQT.

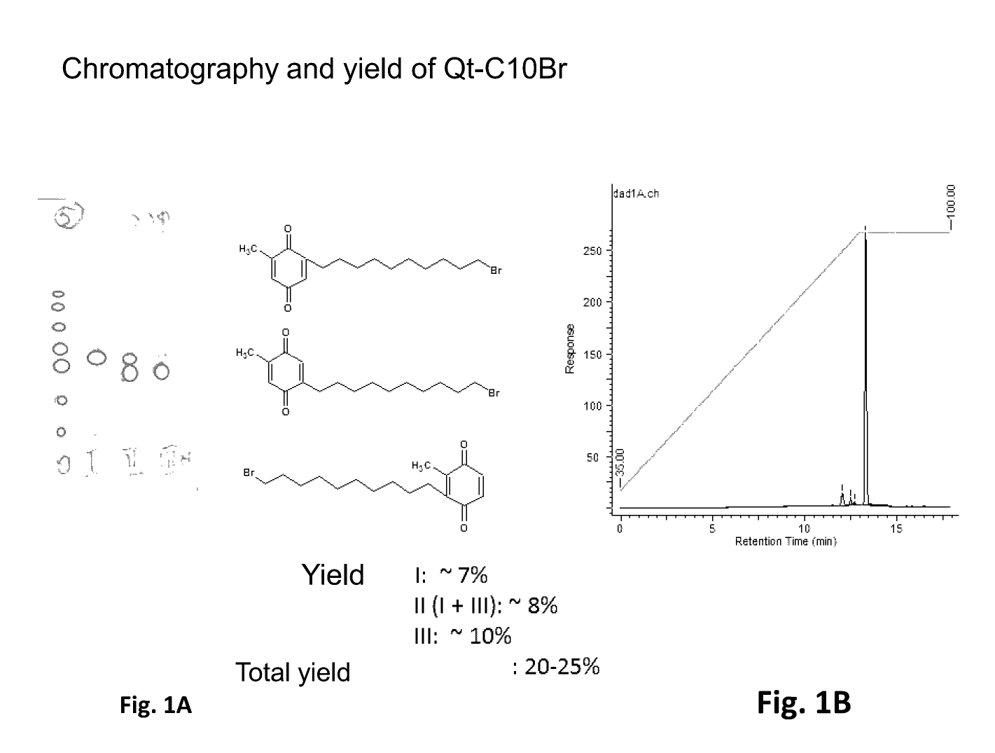

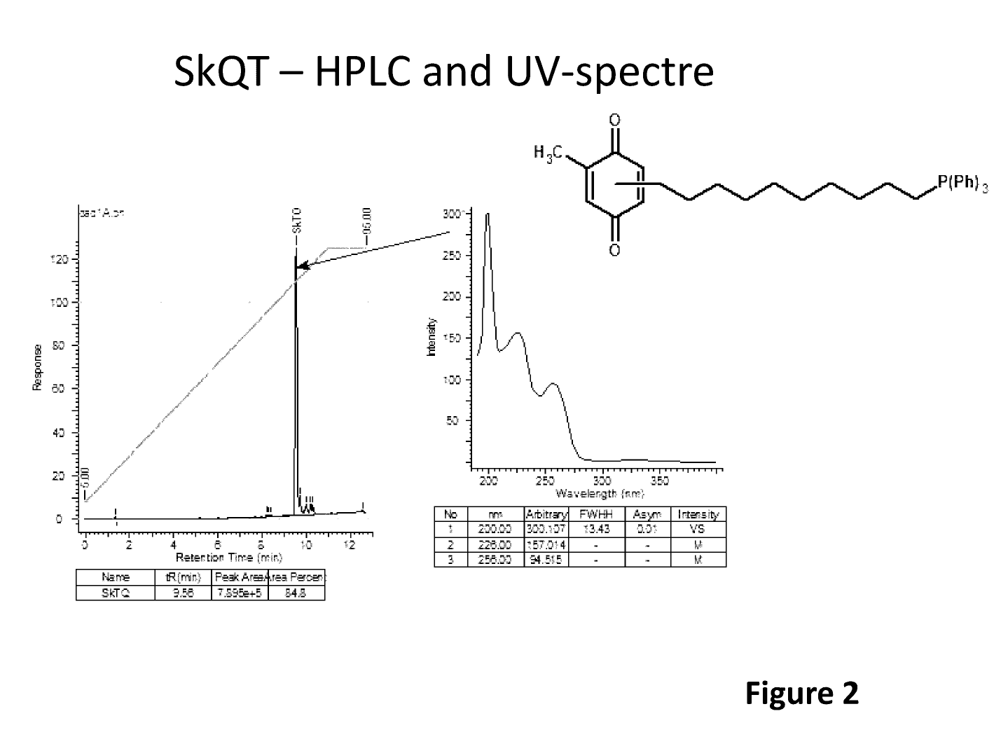

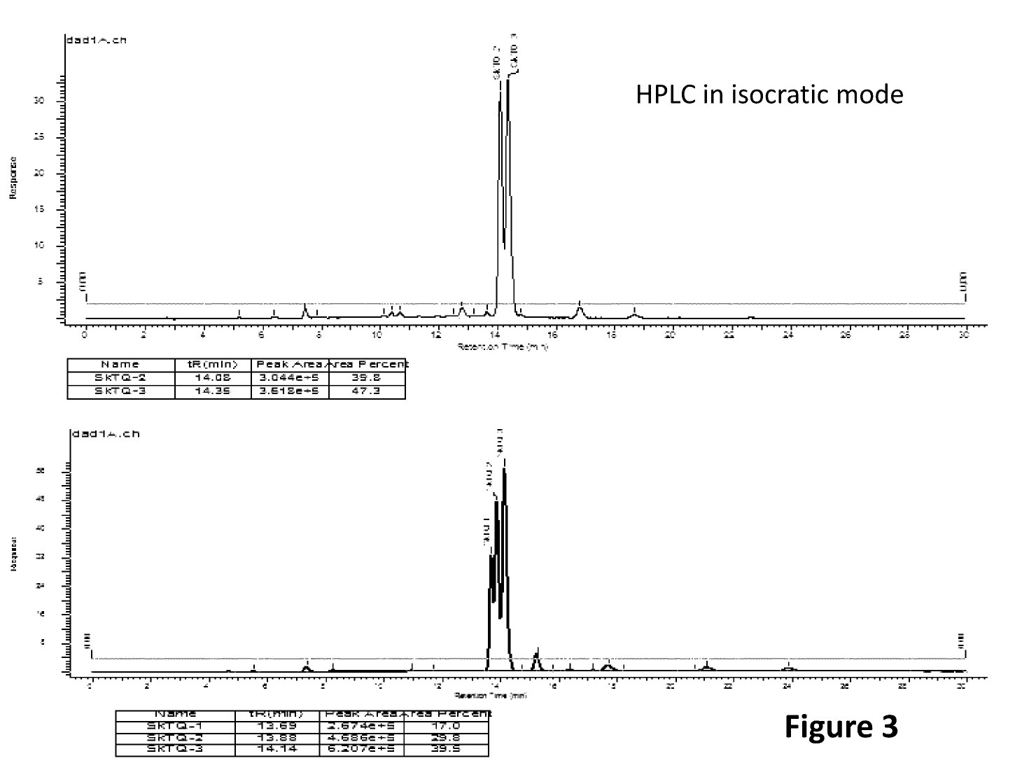

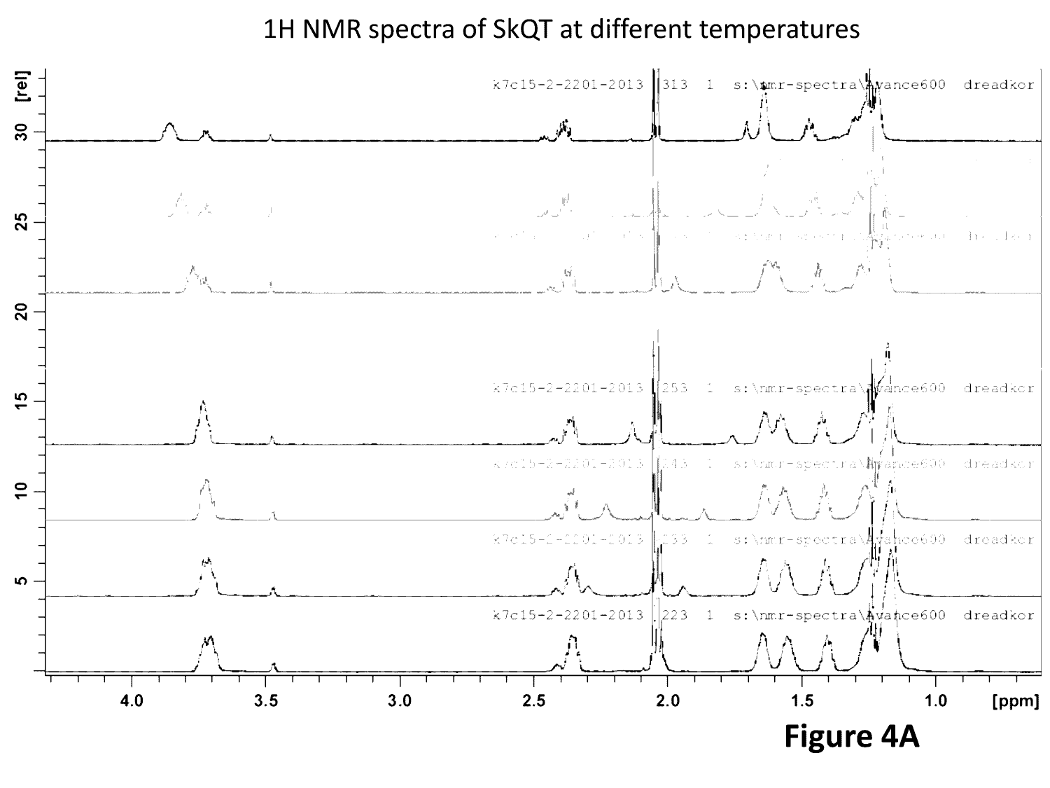

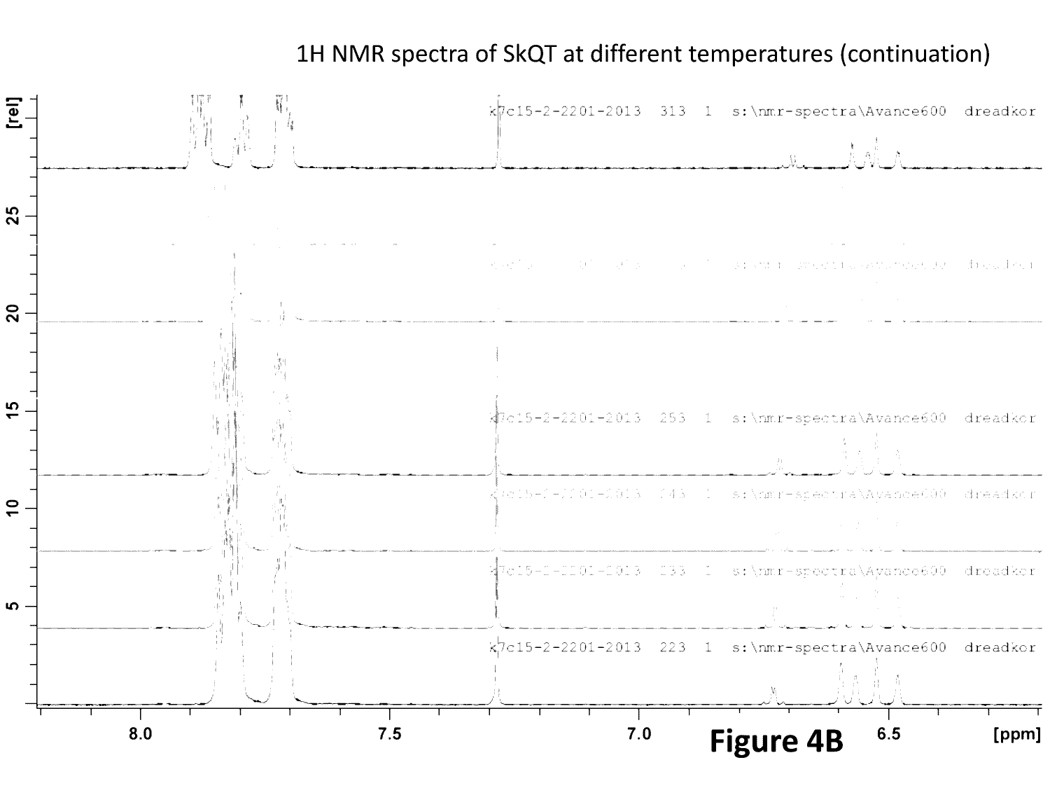

### DETAILED DESCRIPTION OF THE PREFERRED EMBODIMENTS

The invention relates to the fields of pharmacology and medicine, and in particular to mitochondrially-targeted quinones and quinoles and pharmaceutical compositions based on these compounds. The invention provides new MTAs having new antioxidant moieties.

The present invention provides new structures of MTAs comprising derivatives of timoquinone (see formula 1) as antioxidant moieties.

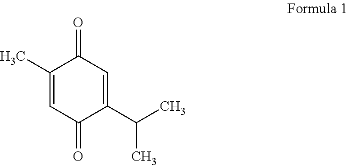

These timoquinone-based MTAs can be described by the general formula 2:

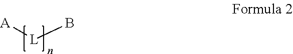

### Wherein:

A is an timoquinone-derived antioxidant moiety of Formula 3:

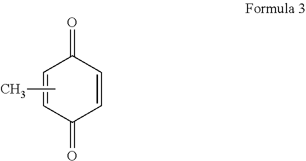

or a reduced form thereof, wherein the CH3 group is attached to any free position of quinone ring;
or A is an timoquinone-derived antioxidant moiety of Formula 4:

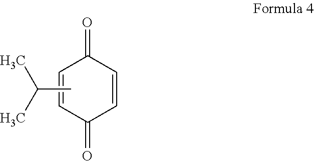

or a reduced form thereof, wherein the isopropyl group is attached to any free position of quinone ring;
or A is an timoquinone-derived antioxidant moiety of Formula 5:

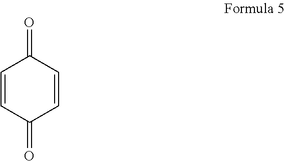

or reduced form thereof;

L is a linker group, comprising: a) a straight or branched hydrocarbon chain optionally substituted by one or more double or triple bond, or ether bond, or ester bond, or C—S, or S—S, or peptide bond; and which is optionally substituted by one or more substituents preferably selected from alkyl, alkoxy, halogen, keto group, amino group; or b) a natural isoprene chain;

n is an integer from 1 to 20; and

B is a targeting group comprising: a) a Skulachev-ion Sk (Sk⁺ Z⁻) wherein: Sk is a lipophilic cation or a lipophillic metalloporphyrin, and Z is a pharmaceutically acceptable anion; or b) an amphiphillic zwitterion.

In some embodiments, timoquinone-based MTAs include SkQT1:

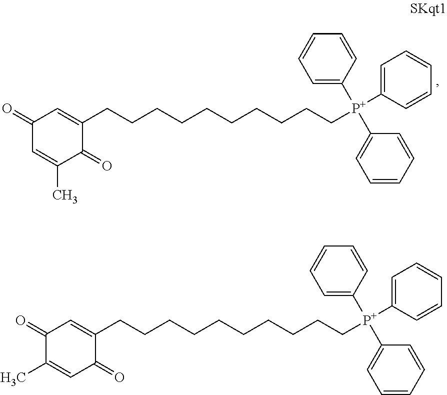 [SMILES: CC1=CC(=O)C=C(CCCCCCCCCC[P+](c2ccccc2)(c2ccccc2)c2ccccc2)C1=O ; CC1=CC(=O)C(CCCCCCCCCC[P+](c2ccccc2)(c2ccccc2)c2ccccc2)=CC1=O]

its isomer SkQT2
SkQT2,
its isomer SkQT3

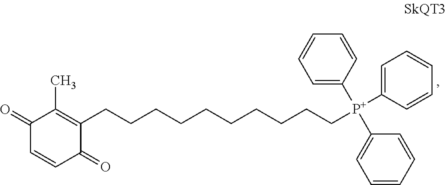 [SMILES: CC1=C(CCCCCCCCCC[P+](c2ccccc2)(c2ccccc2)c2ccccc2)C(=O)C=CC1=O]

and SkQT which is mixture of SkQT1, SkQT2 and SkQT3;
and reduced forms thereof.

In some embodiments, the timoquinone-based MTA includes SkQTP1

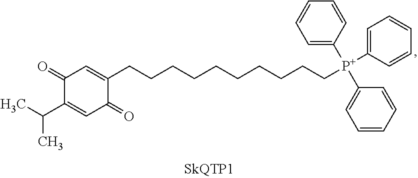 [SMILES: CC(C)C1=CC(=O)C(CCCCCCCCCC[P+](c2ccccc2)(c2ccccc2)c2ccccc2)=CC1=O]

its isomer SkQTP2

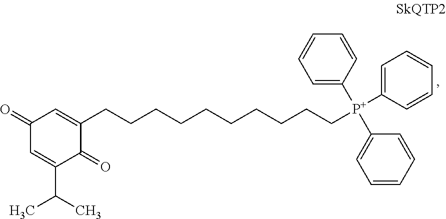 [SMILES: CC(C)C1=CC(=O)C=C(CCCCCCCCCC[P+](c2ccccc2)(c2ccccc2)c2ccccc2)C1=O]

its isomer SkQTP3,

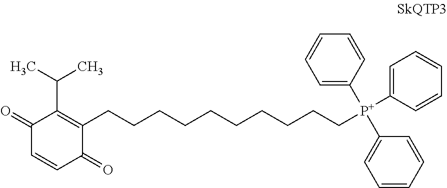 [SMILES: CC(C)C1=C(CCCCCCCCCC[P+](c2ccccc2)(c2ccccc2)c2ccccc2)C(=O)C=CC1=O]

and SkQTP which is a mixture of SkQTP1, SkQTP2 and SkQPT3;
and reduced forms thereof.

In some embodiments, the timoquinone-based MTA includes SkQRT1

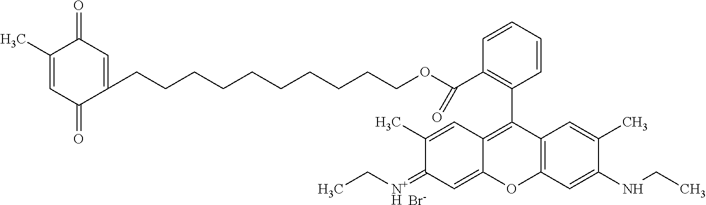

and its isomers SkQRT2, SkQRT3 (analogues for SkQT1-3) and the analogues mixture SkQRT; and reduced forms thereof.

In some embodiments, the MTA is a demethylated analogue of SkQT, termed SkQB:

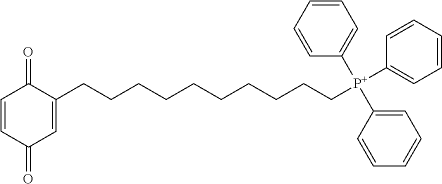 [SMILES: O=C1C=CC(=O)C(CCCCCCCCCC[P+](c2ccccc2)(c2ccccc2)c2ccccc2)=C1]

All of these compounds exist in both oxidized (as shown above) and reduced forms.

In some embodiments, the targeting moiety (B) for the compounds of formula 2 is the lipophilic cation:

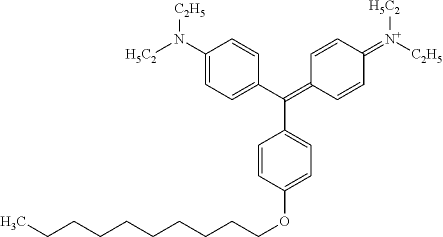

This formula shows, but is not limited to the L-linker decane (i.e., n=an integer from 1-20).

The experiments with the SkQ variants described above showed that SkQT in optimal concentration almost completely prevented hydrogen peroxide formation by mitochondria. Neither very low nor very high concentrations of SkQT have this effect. The window between lowest and highest no effect doses is much wider for SkQT than for SkQ1 (see biological examples 5 and 6). Pharmaceutical formulations and administration

In the methods according to the invention, the compounds described above may be incorporated into a pharmaceutical formulation. Such formulations comprise the compound, which may be in the form of a free acid, salt or prodrug, in a pharmaceutically acceptable diluent (including, without limitation, water), carrier, or excipient. Such formulations are well known in the art and are described, e.g., in Remington's Pharmaceutical Sciences, 18th Edition, ed. A. Gennaro, Mack Publishing Co., Easton, Pa., 1990. The characteristics of the carrier will depend on the route of administration. As used herein, the term “pharmaceutically acceptable” means a non-toxic material that is compatible with a biological system such as a cell, cell culture, tissue, or organism, and that does not interfere with the effectiveness of the biological activity of the active ingredient(s). Thus, compositions according to the invention may contain, in addition to the inhibitor, diluents (including water), fillers, salts, buffers, stabilizers, solubilizers, and other materials well known in the art. As used herein, the term “pharmaceutically acceptable salts” refers to salts that retain the desired biological activity of the above-identified compounds and exhibit minimal or no undesired toxicological effects. Examples of such salts include, but are not limited to, salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, palmoic acid, alginic acid, polyglutamic acid, naphthalenesulfonic acid, naphthalenedisulfonic acid, methanesulfonic acid, p-toluenesulfonic acid and polygalacturonic acid. The compounds can also be administered as pharmaceutically acceptable quaternary salts known by those skilled in the art, which specifically include the quaternary ammonium salt of the formula —NR+Z—, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate). The active compound is included in the pharmaceutically acceptable carrier or diluent in an amount sufficient to deliver to a patient a therapeutically effective amount without causing serious toxic effects in the patient treated. A “therapeutically effective amount” is an amount sufficient to alleviate or eliminate signs or symptoms of the disease or condition being treated. The effective dosage range of the pharmaceutically acceptable derivatives can be calculated based on the weight of the parent compound to be delivered. If the derivative exhibits activity in itself, the effective dosage can be estimated as above using the weight of the derivative, or by other means known to those skilled in the art. In certain applications, an effective dose range for a 70 kg patient is from about 1 ug per patient per day up to about 1 gram per patient per day, or the maximum tolerated dose. In certain preferred embodiments the dose range is from about 100 ug per patient per day to about 100 mg per patient per day. In certain preferred embodiments the dose range is from about 200 ug per patient per day to about 30 mg per patient per day. The dose in each patient may be adjusted depending on the clinical response to the administration of a particular drug. Administration of the pharmaceutical formulations in the methods according to the invention may be by any medically accepted route, including, without limitation, parenteral, oral, sublingual, transdermal, topical, intranasal, intratracheal, or intrarectal. In certain preferred embodiments, compositions of the invention are administered parenterally, e.g., intravenously in a hospital setting. In certain other preferred embodiments, administration may preferably be by the oral route.

The following Examples are intended to further illustrate certain embodiments of the invention and are not intended to limit the scope of the invention.

### SYNTHETIC EXAMPLE 1

Synthesis of [10-(2-methyl-3,6-dioxocyclohexa-1,4-dien-1-yl)decyl]triphenylphosphoniumbromide10-(p-toluquinonyl-5)decyl)triphenylphosphonium bromide, SkQT (3)

The synthesis of SkQT is outlined in Scheme 1.

p-Toluquinone (2-methyl-1,4-benzoquinone,l) was alkylated with a bromdecyl group in a reaction of radical substitution with simultaneous decarboxylation in the presence of bromoundecanoic acid, silver nitrate and ammonium persulfate to afford the substituted quinone (2) with good yield. Quinone (1) and bromoundecanoic acid were provided in equimolecular ratio to avoid formation of bis-alkyl derivatives. The main product of this reaction was p-substituted quinone of formula (2), however there were other positional isomers. Product was purified by column chromatography on silica gel. The eluent used was chloroform and methanol (4:1) or dichlormethane and ethanol (6:1). Fractions containing compound corresponding to mono-bromdecylderivative were collected to introduce into a reaction with triphenylphosphine.

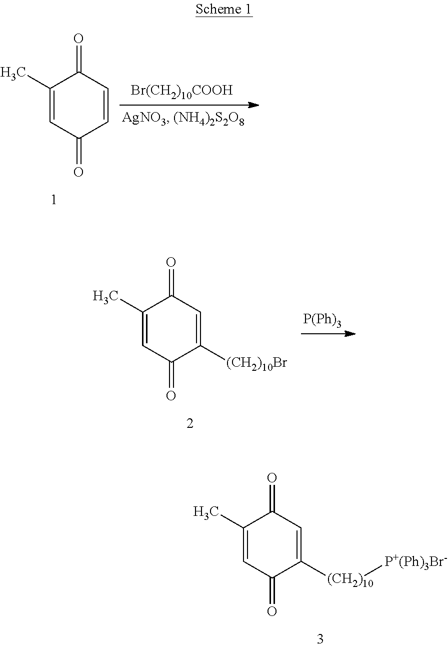

The reaction proceeded for 72 hours in ethanol at 75-80° C. in a tightly closed vessel. After reaction completion the mixture was evaporated and the resulting residue was purified by precipitation by diethyl ether from dichloromethane and finally by column chromatography on silica gel. Target compound was obtained with a yield of 51% and produced a mixture of three isomers of the same mass—523 m/z. It was possible to separate this mixture with help of HPLC, but the yields of isomers were very low. For one of them NMR spectra were recorded and it was shown that this isomer had a structure identical with (3).

A second way of synthesis of compound (3) can be performed according to the following scheme (Scheme 2). It differs from Scheme 1 in the two following steps. 10-(p-Toluquinonyl-5)-decylbromide (2) was reduced by NaBFU in methanol to afford 2-methyl-5(10-bromdecyl)-1,4-hydroquinone (2a) with a yield of 75%. The latter was introduced into a reaction with triphenylphosphine followed by oxidation with oxygen in chloroform solution to produce the target compound (3) possessing the same characteristics as indicated earlier.

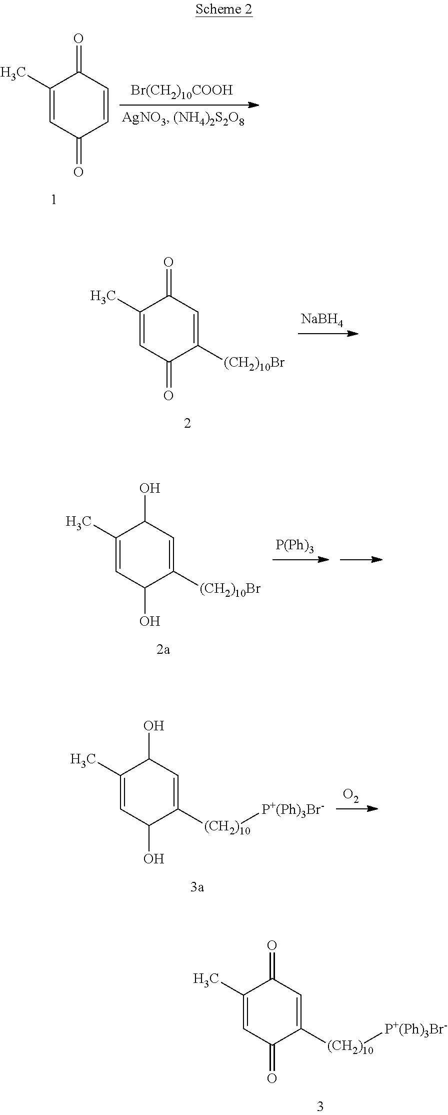

2-Methyl-1,4-benzoquinone (p-toluquinone), 11-bromoundecanoic acid, triphenylphosphine, silver nitrate, ammonium persulfate, solvents were obtained from Acros organics, Fluka, Aldrich, Sigma. TLC was carried out on silica gel 60 F254 plates (Merck), for column chromatography silica gel 60 (0.063-0.2 mm) (Fluka) was used. Reversed-phase HPLC was carried out on Adjilent 1100. Compounds absorbing the UV-light were monitored using a Brumbergchemiscope. Compounds with a triphenylphosphonium group were detected by Dragendorf reagent. Molecular masses were determined by ESI or MALDI TOF mass spectrometry on Bruker instruments. Proton and two-dimensional NMR spectra for solutions of substances in CDCI3 and CD₃OD were recorded on Bruker AV-600NMR spectrometers with operating frequencies 600.13 MHz for protons; and C spectra with decoupling from protons using WALTZ modulation. The residual signals of chloroform (SH 7.27 ppm, 8 c 77.0 ppm) were used as internal standards. Two-dimensional spectra were recorded using standard methods (COSY, in the magnitude presentation; for HSQC and HMBC, ¹JCH⁼

145 Hz, HMBC-3 JCH⁼ 10 Hz, respectively, were used). Chemical shifts are reported in ppm (8) and spin-spin coupling constants are in hertz.

10(2-methyl-1,4-benzoquinonyl-5)-decylbromide, (2).

2-Methyl-1,4-benzoquinone (3.05 g,0.025 mol) was dissolved in 25 ml of the AcCN, then a solution of AgNC<<3 (2.125 g, 0.0125 mol) in 25 ml of H2O was added. The reaction mixture was heated to 60° C. (temperature of solution) and 11-bromundecanoic acid (6.625 g, 0.025 mol) in 25 ml of AcCN and a solution of (NH₄)2S₂0₈ (6.95 g, 0.025 mol) in 25 ml of H₂0 were added dropwise with stirring at 60-70° C. for 3 h. After dilution with water, the mixture was extracted by methylene chloride. The organic layer was washed with water, dried over Na2SC>4 and evaporated in vacuo. The residue was applied to a silica gel column (140×35 mm) using chloroform as eluent. The yield was 5.21 g (61.1%). TLC: Rf (chloroform)=0.82. HPLC: x=7.26 min (0-90% B for 20 min; A: 10 mM H3PO4, B: AcCN). UV (methanol): ?w 254 nm.

10-(2-methyl-1,4-benzoquinonyl-5)decyltriphenylphosphonium bromide, SkQT (3).

Triphenylphosphine (433 mg, 0.165 mol),10-(2-methyl-1,4-benzoquinonyl-5)-decylbromide (563 mg,0.165 mol) and 96% ethanol (1.4 ml) were placed in tightly closed glass vessel and kept at 75-85° C. for 72 h. Addition of diethyl ether to the resulting solution gave a precipitate, then the supernatant was decanted. The residue was dissolved in a minimum amount of dichloromethane and precipitated by diethyl ether again. This procedure was repeated three to four times. The final product was dried under vacuum and purified by column chromatography on silica gel with chloroform-methanol (4:1) as eluent. The yield of (3) was 298.5 mg (30%). TLC: Rf (chloroform-methanol, 4:1)=0.69. HPLC: x=9.56 min (5-95% B for 12 min; A: 0.05% TFA, B: 0.05% TFA in AcCN); xi=13.72 min, x₂=13.92 min, x₃=14.18 min (5-90% B for 30 min; A: 0.05% TFA, B: 0.05% TFA in AcCN). Ratio of isomers (area percent) were different and roughly equal to 1.4; 1.UV (methanol): ?wₓ 200 nm, 226 nm, 256 nm, ESI MS: m/z calcd. For C35H40O2P 523.6/found 523.3. [Ph₃P-5-CioH2o-C6H202-2-CH₃]⁺Br—:

¹H NMR (600 MHz, CDCl3): 1.22 (m, 12H, 6CH₂), 1.46 (quint, 2H, CH₂, J=7.2 Hz), 1.62 (m, 2H, CH₂), 2.04 (d, 3H, CHₓJ=1.4 Hz), 2.38 (m, 2H, CH₂), 3.71 (m, 2H, CH₂), 6.51 (s, 1H, CH_{M6)}), 6.58 (quad, 1H, Ci/_{M}3), J=1.4 Hz), 7.72 (m, 6H, 6a-H_{Ph}), 7.82 (m, 9H, 6P-Hₚₕ+3y-Hₚₕ). ¹³C NMR (150.9 MHz, CDCl₃): 15.54 (CH₃), 22.67 (2CH₂), 29.25 (8CH₂), 118.12-135.12 (18C(_{Ph)}), 132.38 (CHÂ)), 133.68 (CĤ)), 145.75 (C_{M2)}), 149.71 (C_{M5)}), 187.92 (C=0₍₄₎), 188.42 (C=0₍i)).

10-(2-Methyl-1,4-dihydroxyphenyl-5)decybromide (2red).

Fine powder of sodium borohydride (2 g, 0.055 mol) was added under stirring for 15 minutes to solution of 10-(2-methyl˜1,4-benzoquinonyl-5)decylbromide (4 g, 0.012 mol) in 40 ml of methanol. After the reaction end excess NaBFL; was neutralized with 5% HCl, and the reaction mixture was diluted by water and extracted with diethyl ether 2-3 times. The ether solution was washed two times by brine and dried over Na₂S04. After evaporation of solvent the yield of the product was 3.1 g (75%). Purification of (2red) was performed by column chromatography on silicagel using chloroform as eluent.

10-(2-Methyl-1,4-dihydroxyphenyl-5)decyltriphenylphosphonium bromide (3red).

A mixture of 10-(2-methyl-1,4-dihydroxyphenyl-5)decylbromide (1.70 g, 0.005 mol), triphenylphosphine (1.31 g, 0.005 mol) in 10 ml of ethanol was heated at 85° C. for 72 hours in a brown flask tightly closed with Teflon stopper. After completion, the reaction mixture was treated similarly to (3): first by several precipitations by diethyl ether from dichloromethane, finally by column chromatography on silica gel (280×35mm) with the use of dichloromethane-ethanol (6:1) as an eluent. The product (3red) was obtained as a light brown powder with a yield of 2.4 g (79.3%). Oxygen was passed through the solution of (3red) in chloroform at room temperature affording (3).

Preparation of 10-(2-methyl-1,4-benzoquinonyl-5)decylbromide (2a,p-Isomer) and 10-(2-methyl-1,4-benzoquinonyl-6)decylbromide (2b, m-Isomer)

To 4.88 g (0.04 mol.) of 2-methyl-1,4-benzoquinone and 3.4 g (0.02 mol.) of silver nitrate in a mixture of 130 ml of acetonitrile and 90 ml of water stirring at 60° C., 10.6 g (0.04 mol.) of 11-bromoundecanoic acid in 130 ml of acetonitrile and 9.1 g (0.04 mol.) of ammonium persulfate in 90 ml of water were added drop by drop during 3 h. The stirring was continued at the same temperature for 12 h after all reagents were added. Then acetonitrile was evaporated from the reaction mixture and a residue was extracted by diethyl ether (3×150 ml), combined ether layers were washed sequentially with IN aqueous HCl, water, 5% aqueous sodium bicarbonate and water, then dried with anhydrous sodium sulfate and evaporated to dryness. The residue was separated on a silica gel column using methylene chloride as eluent giving 3 fractions of 2 with a total yield of 20.7% (2.82 g): fraction 1 contains 10-(2-methyl-1,4-benzoquinonyl-5)decylbromide (2a, p-isomer): 750 mg (5.5%); HPLC: ×13.0 min (gradient: from 35 to 95% of acetonitrile in 0.1% aqueous TFA during 13 min); ESI-MS: m/z ([M+MeCN] found/calculated: 382/382; TLC: Rf=0.53 (chloroform); fraction 3 contains 10-(2-methyl-1,4-benzoquinonyl-6)decylbromide (2b, m-isomer): 1.243 g (9%); HPLC: ×13.0 min (gradient: from 35 to 95% of acetonitrile in 0.1% aqueous TFA during 13 min); ESI-MS: m/z ([M+MeCN] found/calculated: 382/382; TLC: Rf=0.47 (chloroform); fraction 2 contains a mixture of 2a and 2b.

### SYNTHETIC EXAMPLE 2

### Synthesis of (2-methyl-1,4-benzoquinonyl-5)decyl ester of rhodamine 19(R1) (SkQRT1) (5)

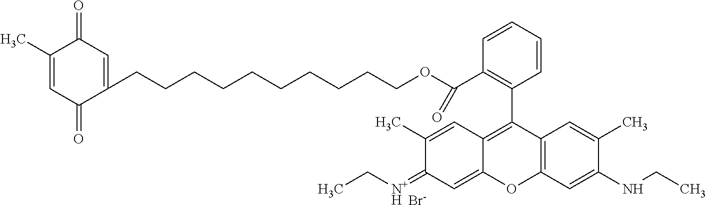

### Structure of SkQRT1

The synthesis of SkQRT1 was carried out according to Scheme 3

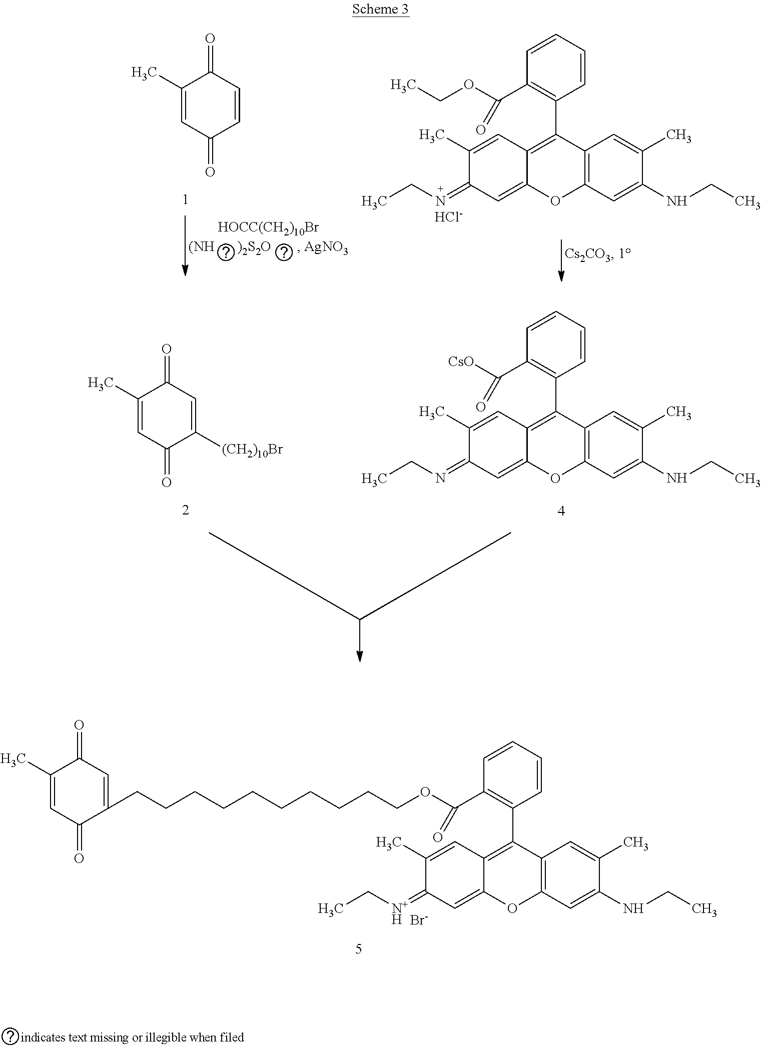

Cesium salt of rhodamine 19 (4) was prepared for reaction with bromo-derivative2, as shown:

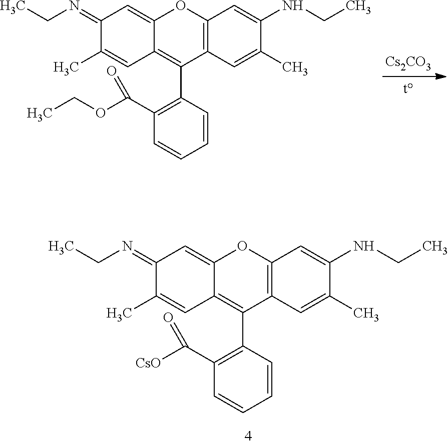

Compound (4) was prepared by adding of 2M aqueous cesium carbonate (4 eq.) to a methanol solution of ethyl ester of rhodamine 19, the mixture was heated up to boiling, then was cooled, a product was isolated by filtration with a yield of 75%.

Cesium salt of rhodamine 19 was condensed with bromo-derivative (2):

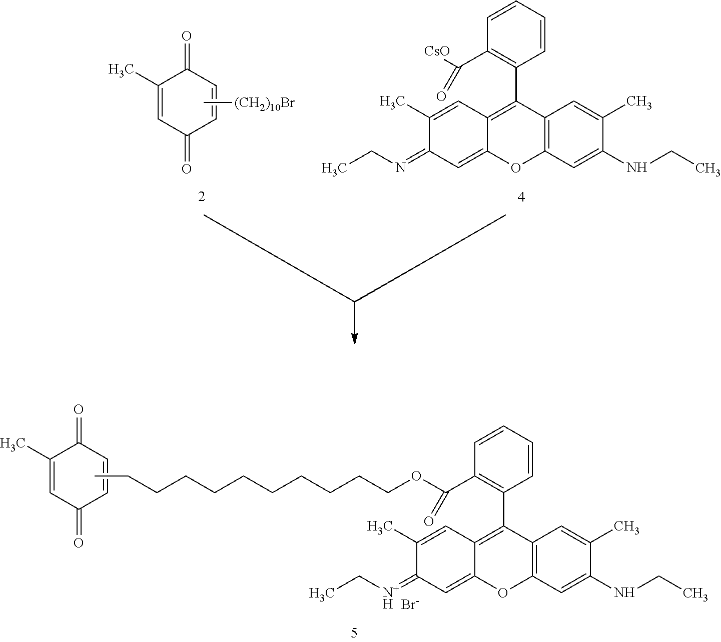

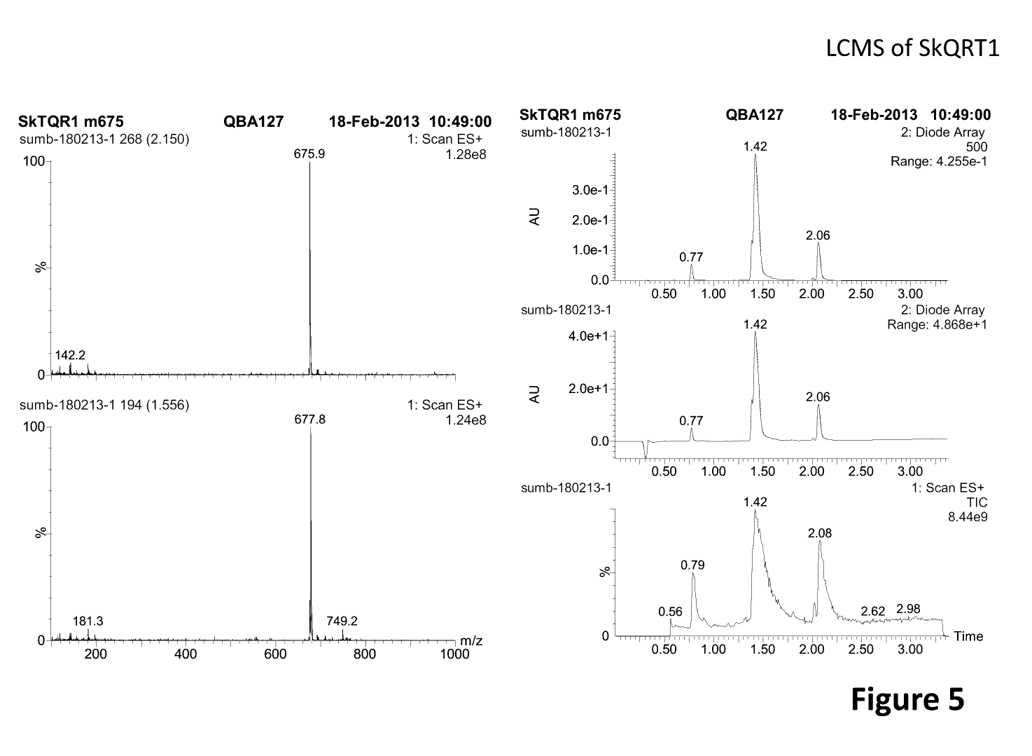

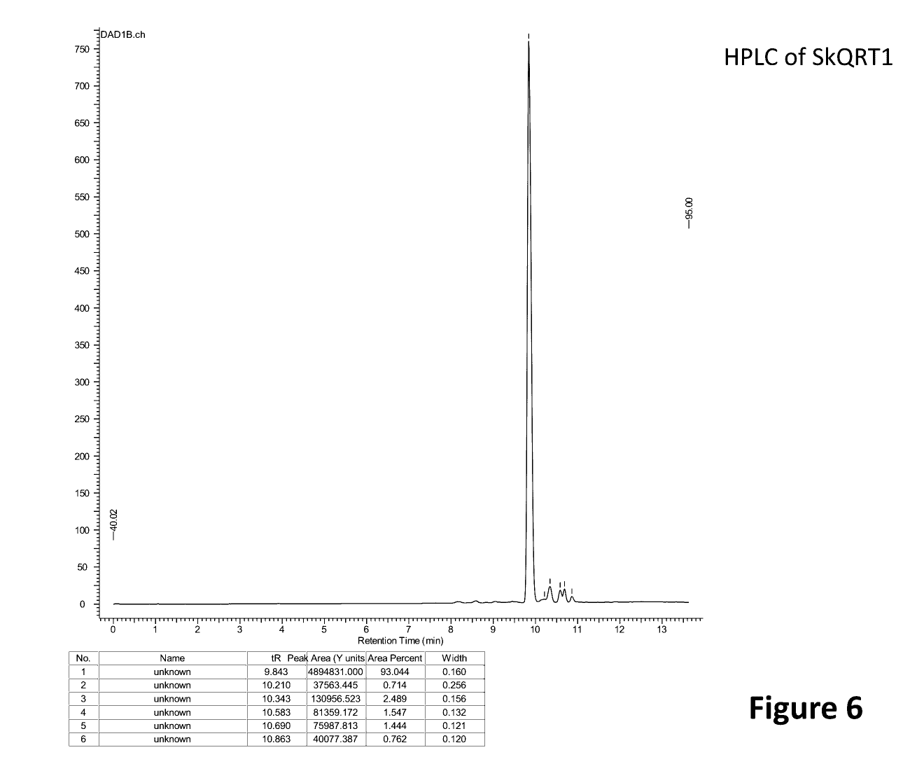

A mixture of equimolecular quantities of cesium salt 4 and bromo-derivative 2 in DMF was kept at 60° C. for 72 h. The resulting product was precipitated by adding diethyl ether, a precipitate was isolated by decantation, then dissolved in chloroform and oxidized by passing oxygen through this solution. SkQRT1 was isolated by column chromatography on silica gel using a mixture of dichloromethane and ethanol (7:1, v/v) as eluent. The compound gave a single peak on TLC and HPLC (×10.2 min, gradient: from 40 to 95% of acetonitrile in 0.1% aqueous TFA during 11 min), LC-MS: m/z): 675.9/675.4, ([M+3H]+ found/calculated): 677.4/678.0 (M corresponds to reduced form of SkQRT1) (FIG. 5).

Reaction of individual p-isomer of bromdecyl derivative 2a with rhodamine cesium salt was performed analogously as described for 2.

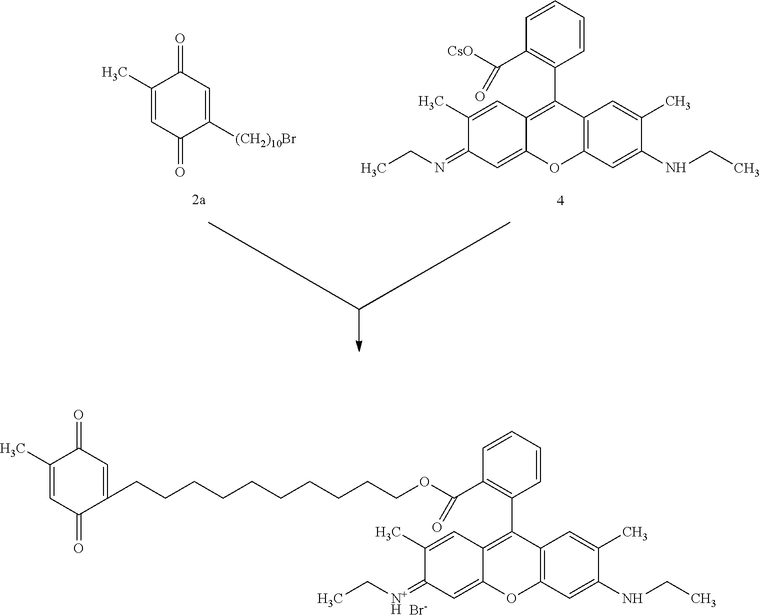

### Preparation of Rhodamine 19 Cesium Salt (4)

13.5 g (0.03 mol.) of rhodamine 19 ethyl ester was dissolved in 250 ml of methanol, 39.12 g (0.12 mol.) of cesium carbonate in 60 ml of water (2 M solution) were added and a mixture was heated to boiling, then cooled, and a product was isolated by filtration giving 12 g (75%) of rhodamine 19 cesium salt (4).

Preparation of 10-(2-methyl-1,4-benzoquinonyl-5)-decyl- and 10-(2-methyl-1,4-benzoquinonyl-6)-decyl-esters of Rhodamine 19 (Mixture of Isomers, 5) (SkQRT1)

827 mg (2.42 mmol.) of 2 were dissolved in 7.5 ml of DMF and added to 1.32 g (2.42 mmol.) of rhodamine 19 cesium salt (4) loaded into a vessel with a threaded lid and the mixture was kept for 72 h at 65° C. The resulting product was precipitated by adding diethyl ether, and a precipitate was isolated by decantation, dissolved in chloroform and oxidized by passing oxygen through the solution and the product was isolated by the column chromatography on silica gel using a mixture of methylene chloride and ethanol (7:1, v/v) as eluent giving 670 mg (41%) of SkQRT1 (mixture of isomers): HPLC: ×10.2 min (gradient: from 40 to 95% of acetonitrile in 0.1% aqueous TFA during 11 min), LC-MS: m/z ([M+H]+ found/calculated): 675.9/675.4, ([M+3H]+ found/calculated): 677.8/677.4.

Preparation of 10-(2-methyl-1,4-benzoquinonyl-5)-decyl ester of rhodamine 19 (SkQRT1): 340 mg (1 mmol.) of 2a was dissolved in 3 ml of DMF and added to 546 mg (1 mmol.) of rhodamine 19 cesium salt (4) loaded into a vessel with a threaded lid and the mixture was kept for 72 h at 65° C. The resulting product was precipitated by adding diethyl ether to the reaction mixture after cooling and isolated by column chromatography on silica gel using a mixture of methylene chloride and ethanol (7:1, v/v) as eluent giving SkQRT1: HPLC: ×10.2 min (gradient: from 40 to 95% of acetonitrile in 0.1% aqueous TFA during 11 min), m/z ([M+H]+ found/calculated): 675.9/675.4.

### SYNTHETIC EXAMPLE 3

### Synthesis of 10-(1,4-benzoquinonyl-2)decyltriphenylphosphonium bromide, SkQB (7)

The synthesis of SkQB is outlined in Scheme 4./?-Benzoquinone (1,4-benzoquinone) was obtained from hydroquinone by oxidation with kalium bromate in water in the presence of sulfonic acid at 50° C. The yield of the product was 87.7%. p-Benzoquinone prepared was alkylated with bromundecanoic acid in a reaction of radical substitution with simultaneous decarboxylation in presence of silver nitrate and ammonium persulfate to produce bromdecyl substituted quinone (6) with a yield 50%. 1,4-Benzoquinone and 11-bromundecanoic acid were provided in equimolar ratio. A reaction was carried out in mixture of acetonitrile—water (3:4) at 60-65° C. Product was purified by column chromatography on silica gel. The eluent used was chloroform. In addition, from the reaction mixture the starting compound of 1,4-benzoquinone was isolated in quantity of 15%. Fractions containing a compound corresponding to mono-bromdecylderivative of benzoquinone (6) were collected to introduce into a reaction with triphenylphosphine.

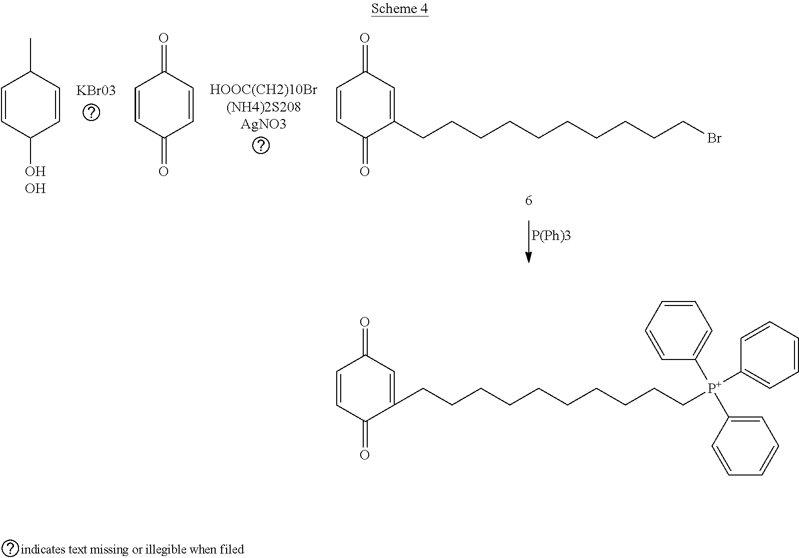

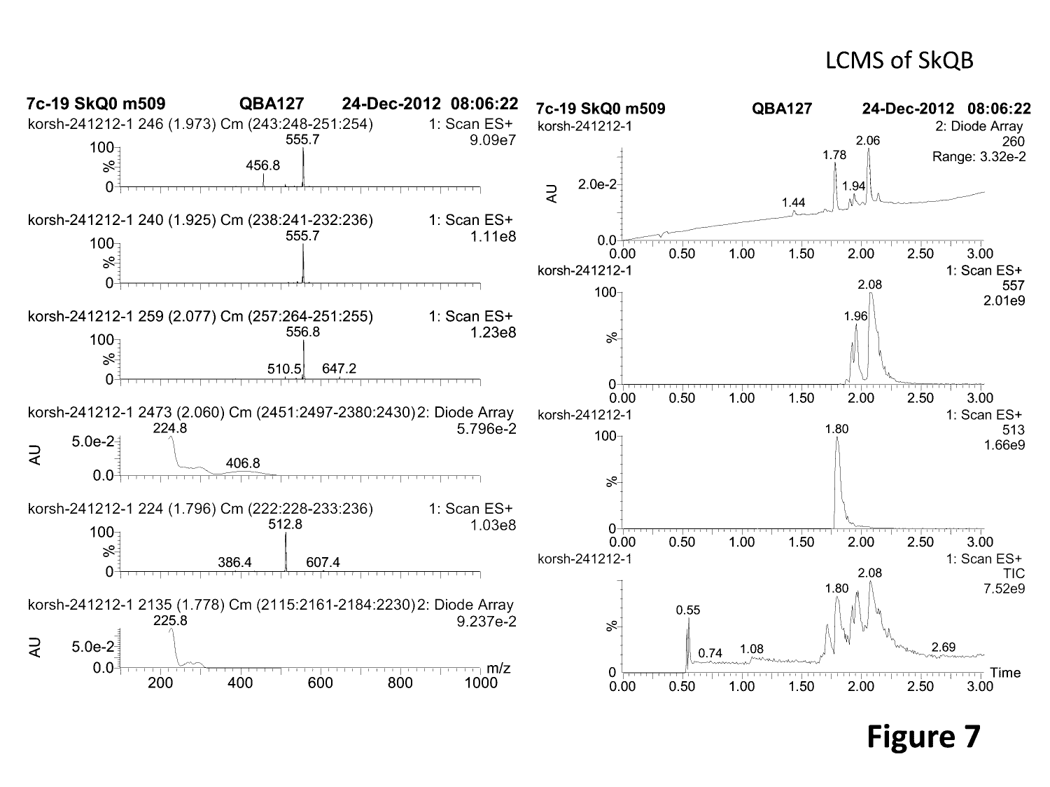

The reaction proceeded for 72 hours in ethanol at 75-80° C. in a tightly closed vessel. After reaction completion the mixture was evaporated and the resulting residue was purified by precipitation by diethyl ether from dichloromethane and finally by column chromatography on silica gel using dichloromethane-ethanol (5:1). Target compound (7) was obtained with a yield 71% and was an individual compound of the mass—512 m/z (See FIG. 7).

10-(1,4-benzoquinonyl-2)-decylbromide, (6).

1,4-Benzoquinone (3.24 g,0.03 mol) was dissolved in 30 ml of AcCN, then a solution of AgNO₃ (2.67 g, 0.015 mol) in 40 ml of H2O was added. The reaction mixture was heated to 60° C. (temperature of solution) and 11-bromundecanoic acid (7.95 g, 0.03 mol) in 30 ml of AcCN and a solution of (NH₄)₂S208 (8.34 g, 0.03 mol) in 40 ml of H₂0 was added dropwise with stirring at 60-70° C. for 3 h. After dilution with water, the mixture was extracted by methylene chloride. The organic layer was washed with water, dried over Na2S04 and evaporated in vacuo. The residue was applied to a silica gel column (140×35 mm) using chloroform as eluent. The yield was 4.92 g (50.0%). TLC: R_{f} (dichloromethane) 0.62.

10-(1,4-benzoquinonyl-2)decyltriphenylphosphoniumbromide, SkQb (7).

Triphenylphosphine (2.62 g, 0.1 mol), 10-(1.4-benzoquinonyl)-decylbromide (3.28 g, 0.1 mol) and 96% ethanol (12 ml) were placed in a tightly closed glass vessel and kept at 75-85° C. for 72 h. Addition of diethyl ether to the resulting solution gave a precipitate, then the supernatant was decanted. The residue was dissolved in a minimum amount of dichloromethane and precipitated by diethyl ether again. This procedure was repeated three to four times. The final product was dried under vacuum and purified by column chromatography on silica gel with dichloromethane-ethanol (5:1) as eluent. M=512.

### SYNTHETIC EXAMPLE 4

### Synthesis of decyltriphenylmethane Derivative (8)

Brilliant green contains a triphenylmethane aromatic system instead of the xantene one of rhodamine B. N,N′-diethylaminotriphenylmethane is more similar to rhodamine 19 because of the presence secondary amino functions.

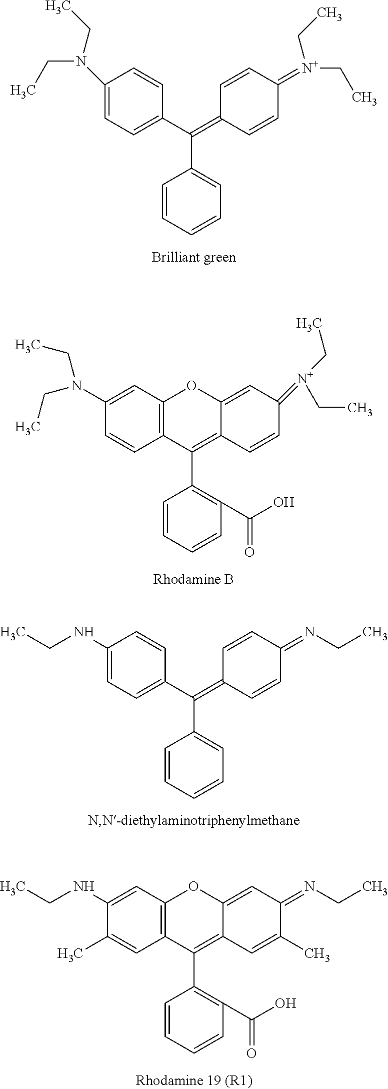

The goal of our work was to synthesize two decyl derivatives of triphenylmethane (8, 9):

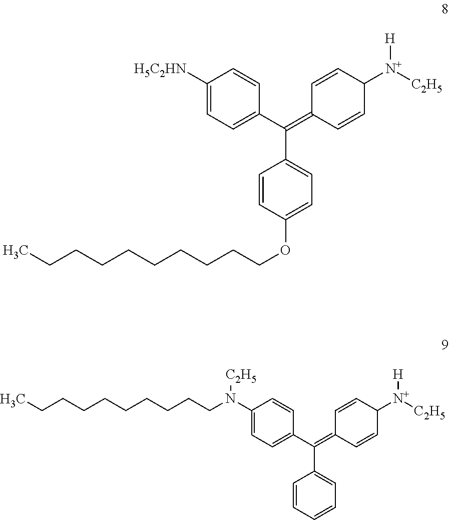

The synthesis of (8) was performed according to scheme 5. Synthesis of the decyl derivative was carried out starting from diethylaniline and p-decyloxybenzaldehyde with the following oxidation to yield 46.1% of compound (8).

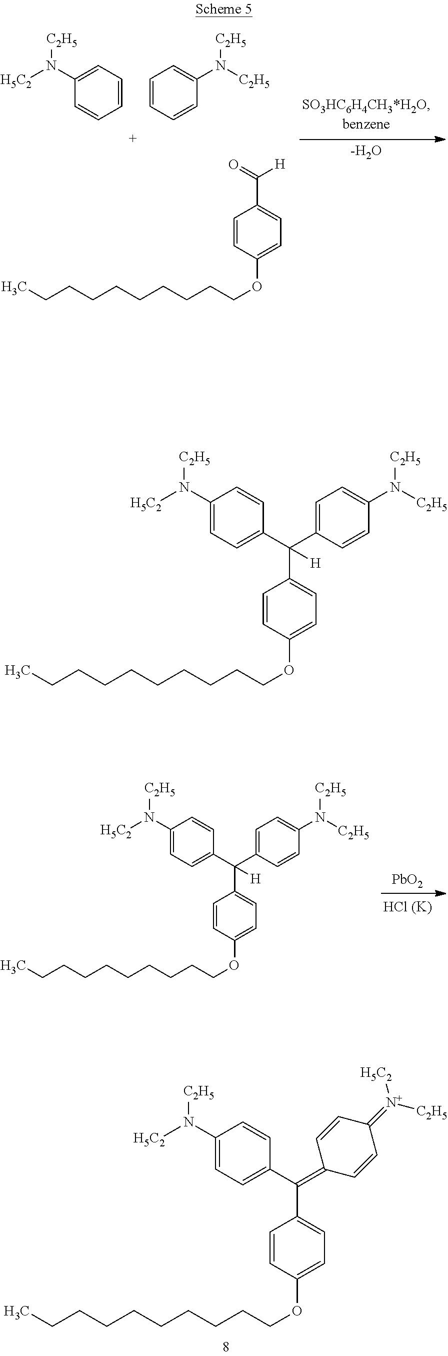

The first step for preparation of 9 was reaction of ethylaniline with benzaldehyde in the presence of toluolsulfonicacid by azeotropic distillation with benzene to produce the compound (8a), as shown at scheme 6. Then its oxidation by PbO₂ in acid medium gave (8b), which was purified by column silica gel chromatography.

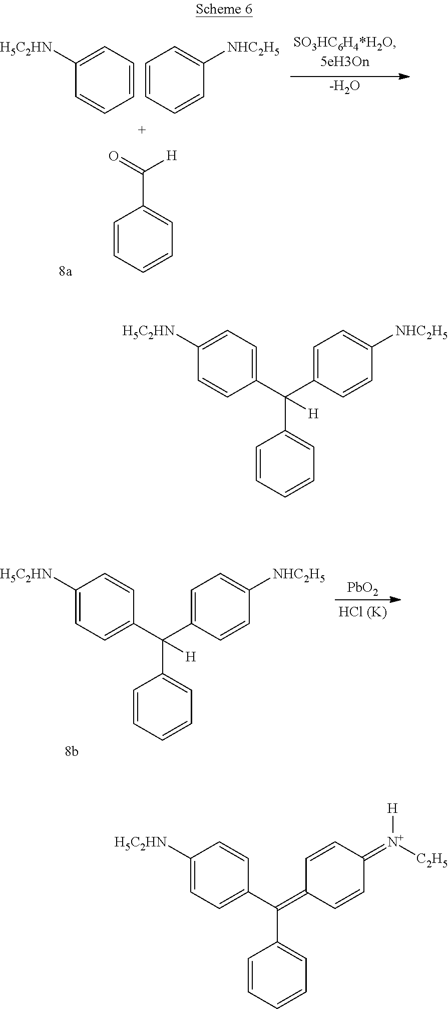

Synthesis of O-decylderivative of triphenylmethane (8)

p-Oxydecylbenzaldehyde (100 mg, 0.38 mmol), N,N-diethylaniline (227.5 mg, 1.53 mmol), p-toluolsulphonic acid (72.5 mg, 0.38 mmol) and benzene (1.5 ml) were refluxed with a device of Dean-Stark. After completion of the reaction, the mixture was warmed at 77° C. in the presence of molecular sieve 4A, then the mixture was evaporated and extracted by dichloromethane. The organic phase was washed with a 10% solution of sodium bicarbonate and brune. After evaporation of solvent the yield was 24.2%. Plumbumdioxyd (28 mg, 0.12 mmol) was added to the aqueous solution (3 ml) of the product obtained (28.6 mg, 0.094 mmol) and reaction mixture was kept 12 hours at ambient temperature with mixing with 3 drops of HCl. The compound was extracted by dichloromethane and was purified on silica gel column using as eluent dichloromethane-methanol, 20:1. The yield was 46.1%. M=541.6

### SYNTHETIC EXAMPLE 5

### New Method of Synthesis of SkQ1

The previously described procedures for the synthesis of SkQ1 were based on direct alkylation of PPh3 with the corresponding alkyl bromide. The reaction proceeds via classic nucleophilic substitution (Br̂PPh₃) mechanism. As is well known, bromide is a quite good leaving group, but not one of the best (for example, iodide, mesylate, tosylate, trifluoromethanesulfonate etc). So, due to not very high activity of both starting materials (Br as a leaving group in alkyl bromide and PPI13 as a nucleophile agent) the substitution (Br->PPli3) reaction takes 72 h (or even more) keeping at a temperature not less than 80-90° C. in order to achieve a satisfactory conversion of the reagents. On the other hand, such conditions seem to be too rigid in this particular case and induce many side reactions including gradual decomposition of the target product in the mixture during the reaction course. As a result, the yield of the product is very low (−20%) and its purification is rather difficult and takes many efforts.

The main idea of our modification is a generation of an alkyl-derivative with a better cleaving group than bromide which would be much more active in nucleophilic substitution reaction with PPI13 (or any other nucleophile, such as other phosphines, amines, etc.). A classic manner to activate any alkyl bromide (or chloride) is its “in situ” conversion to the corresponding iodide by addition of KI (or Nal) as a catalyst into the reaction mixture with nucleophile. Using this approach we found that in our case the reaction proceeds at a temperature of 60° C. (or even less). The possibility of such decreasing temperature in comparison with 80-90° C. needed in previous procedures is important and allows alkylation to proceed without any side reactions, in nearly quantitative yield. It should be noted that in our case KI (or Nal) doesn't work in catalytic amount, because the finally produced iodide-anion is strongly associated with the phosphonium-cation. So, in order to achieve full reaction completion, the starting alkylbromide and KI (or Nal) should be used at least in 1:1 mole ratio. Increasing alkylbromide/KI ratio to 1:2 (or more) accelerates the reaction process. Following the above procedure the product is produced as an iodide. The iodide-ion can be easily changed in water/alcohol media to any other anion, such as bromide, chloride, phosphate, mesylate, or sulfate.

Previously described procedures for the synthesis of SkQ1 require the presence of oxygen or any oxidative reagents in the reaction mixture. Probably, this can be explained by the necessity to oxidize the reduced form of the quinone moiety, which is produced at high temperature as a main side-product. However, there is a drawback in using oxygen or any oxidative reagents in the alkylation reaction, because one of the starting materials, PPI13, can be easily converted to PPI13O. Thus, in the modified procedure we applied an inert atmosphere and anhydrous and degassed solvent.

Suitable solvents for reaction are polar and aprotic ones (the best being acetonitrile; and also acetone, DMF, DMA, HMPA, and other similar solvents). The use of xylene, toluene, benzene, DCM, CHCI3 (and similar others) leads to elimination of side-reactions instead of nucleophilic substitution.

In addition, all manipulations should be carried on without light excess. For example, flasks, funnels for extraction, and chromatography column can be covered with aluminum foil.

### Last Step of Modified Procedure.

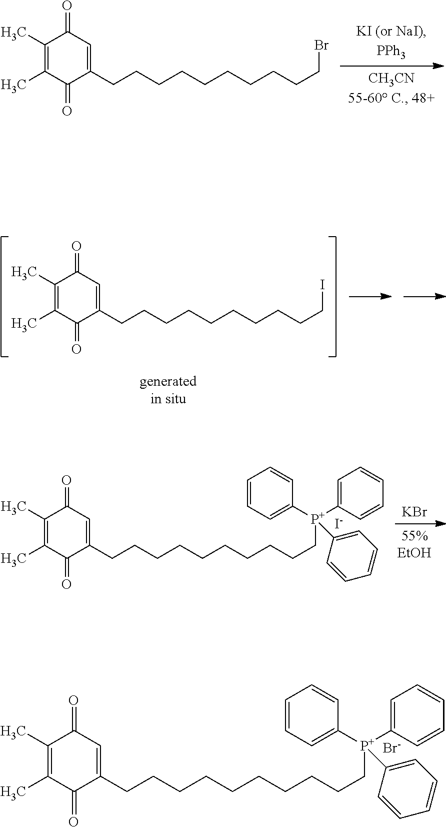

5-(10-Bromodecyl)-2,3-dimethylbenzo-1,4-quinone (40 g, 113 mol), triphenylphosphine (35.4 g, 0.135 mol) and sodium iodide (33.8 g, 0.225 mol) were dissolved in anhydrous acetonitrile (800 mL) in a 2L round bottom flask equipped with an inlet tube, reflux condenser and magnetic stir-bar. The solution was degassed and filled with argon and stirred at 60° C. for 48 h under an argon atmosphere. The solvent was removed in vacuo. Chloroform (1000 mL) was added to the residue. The precipitate was filtered off. The solution was concentrated to 250 mL and placed onto a silica gel column. Eluting with 4% methanol-chloroform mixture followed by evaporation of combined fractions containing the target product produced [10-(4,5-dimethyl-3,6-dioxocyclohexa-1,4-dien-1-yl)decyl](triphenyl)phosphonium iodide (quantitative yield) which was used for the next step. The above product and NaBr (˜30 g) were dissolved in 55% aqueous EtOH (600 ml). The solution was stirred for 30 min under reflux, evaporated to 50% of volume and extracted with CHCI3 (2×75 mL). The combined organic extract was evaporated and the residue was subjected to the repeated procedure (4 times). The product was purified on silica gel column (gradient from 2 to 10% methanol-chloroform) to give the target product, SkQ1 as bromide. Yield 44.7 g (96-99% purity; 64%) yield (yield can be increased) as a deep orange glass-like mass.

### BIOLOGICAL EXAMPLE 1

### Biological Activity of SkQT

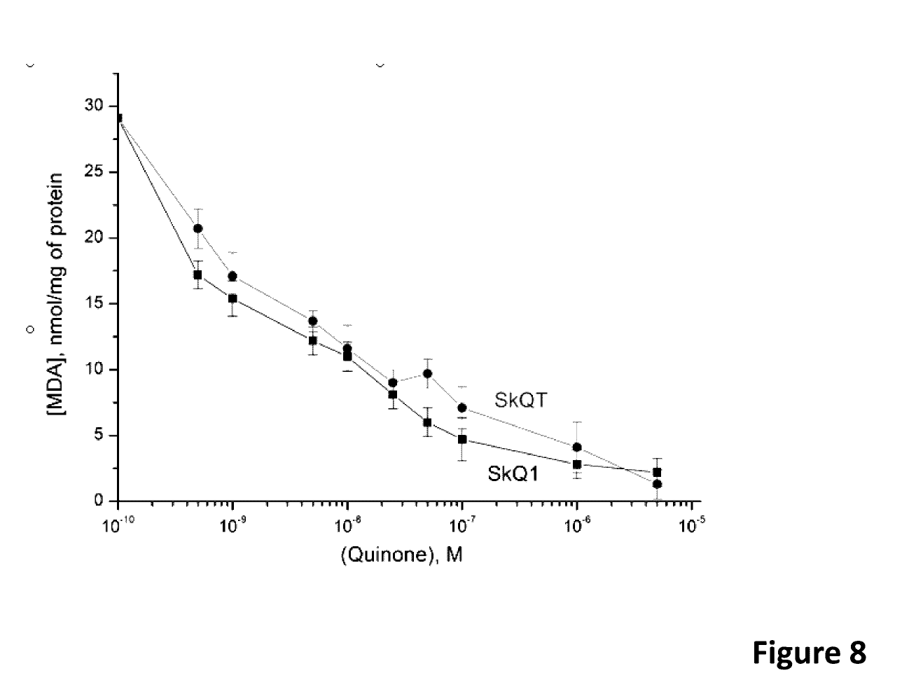

Several compounds according to the invention were synthesized as described above and tested for biological activity as described in Antonenko et al., (Biochem. (Mosc.) (2008) 73:1273-1287). SkQT successfully protected mitochondria from oxidative damage. This was demonstrated by the reduction of the amount of malondialdehyde (MDA) concentration in mitochondria supplemented with certain concentrations of SkQT (see FIG. 8).

### BIOLOGICAL EXAMPLE 2

### Reduction of SkQT by Mitochondrial ETC

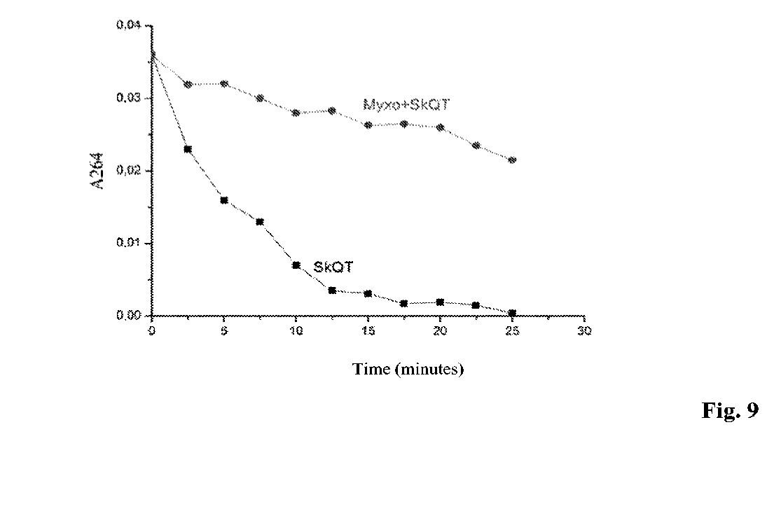

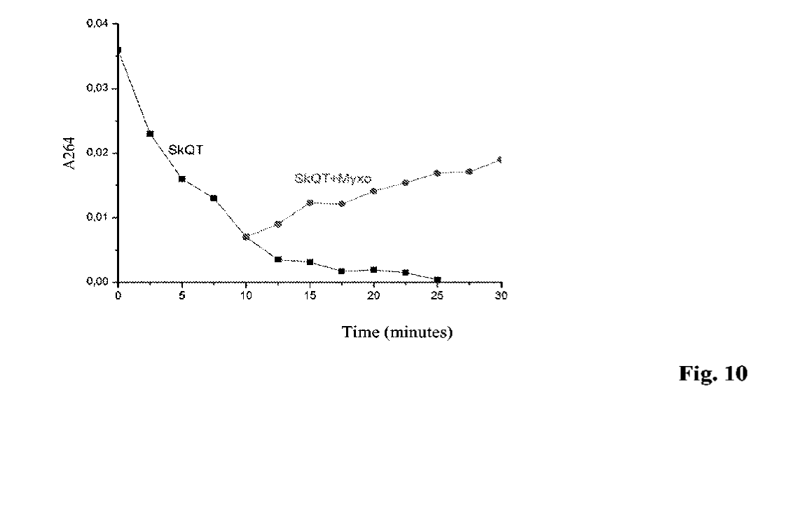

An important feature of MTAs is their ability to be reduced by electron transport chain (ETC) of mitochondria (see Antonenko et al, (Biochem. (Mosc.) (2008) 73:1273-1287 for details). The hypothesis that SkQT can be reduced by mitochondrial ETC was proved by the following experiment on mitochondria isolated from rat heart. Isolated mitochondria (0.05 mg/ml by protein content) were supplemented with 5 mM succinate, 2 uM rotenone, 100 nM SkQT and optionally with 1 uM myxothiasole. The results (FIG. 9) show that SkQT can be reduced by mitochondria. This fact was also proved in the experiment when myxothiasole was added at 10 min point to the mixture. As shown in FIG. 10, this inhibitor prevented further reduction of the quinone form of SkQT.

### BIOLOGICAL EXAMPLE 3

### SkQT Prevents Peroxide-Induced Cell Death

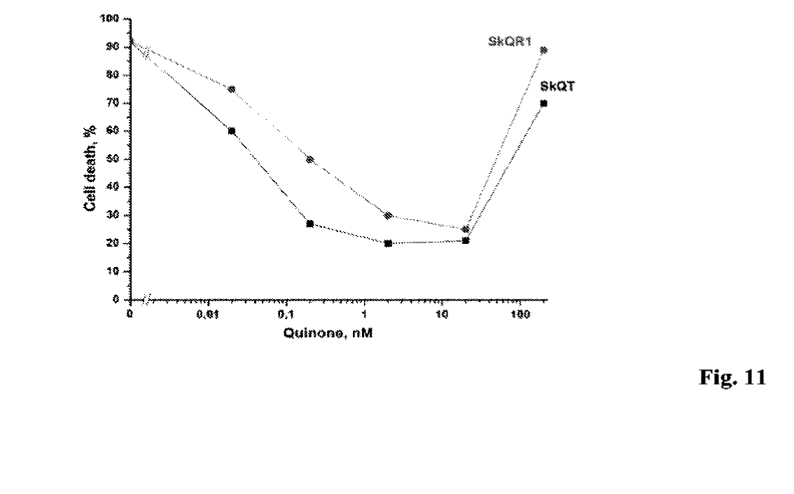

In the next experiment we proved that SkQT is very efficient in prevention of peroxide-induced cell death of human fibroblasts. Its activity was compared to the one of the most efficient MTA-SkQR1. The experiment was performed according to Antonenko et al., (Biochem. (Mosc.) (2008) 73:1273-1287 and the results are shown in FIG. 11.

### BIOLOGICAL EXAMPLE 4

### Dose-Dependent Protection from Peroxide-Induced Cell Death

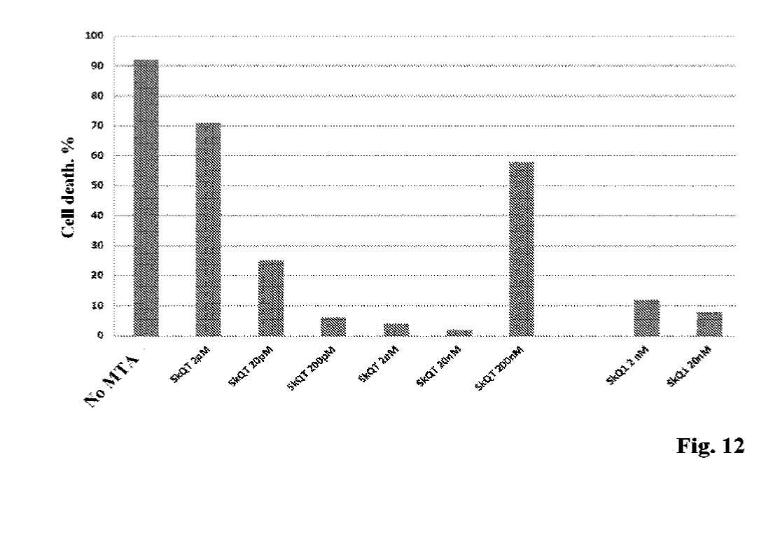

In the next experiment protection of human fibroblasts from H2C<<2-induced cell death by different dosages of SkQT was demonstrated. The experiment was performed according to Antonenko et al., (Biochem. (Mosc.) (2008) 73:1273-1287. (see FIG. 12).

### BIOLOGICAL EXAMPLE 5

### SkQT Prevents H₂O₂ by Y. lipolytica Mitochondria

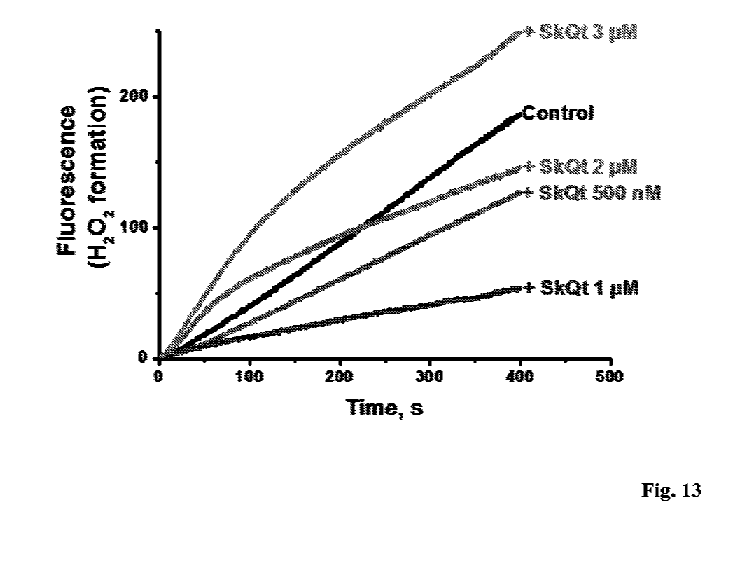

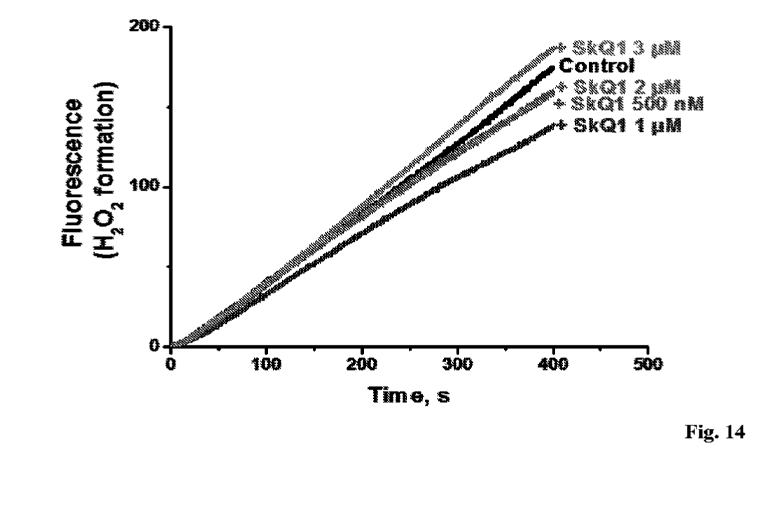

In the next experiment it was shown that certain concentrations of SkQT dramatically decrease the rate of H₂0₂ formation by Y. lipolytica mitochondria. The results are presented on FIG. 13. The experiment was performed in the following conditions: incubation medium contained 0.6 M mannitol, 20 mM Tris-succinate,0.5 mM EGTA, 0.2 mM Tris-phosphate, pH 7.2, 2 uM Amplex red, horse radish peroxidase (5 U) and mitochondria corresponding to 0.1 mg protein. SkQt was added into the incubation medium. Measurements were made using absorption/emission 530/590 nm. It must be noted that the effect of SkQ1 studied in the same conditions was considerably lower (see FIG. 14).

### BIOLOGICAL EXAMPLE 6

### High Concentrations of SkQT Fail to Promote H₂O₂ Formation by Rat Liver Mitochondria

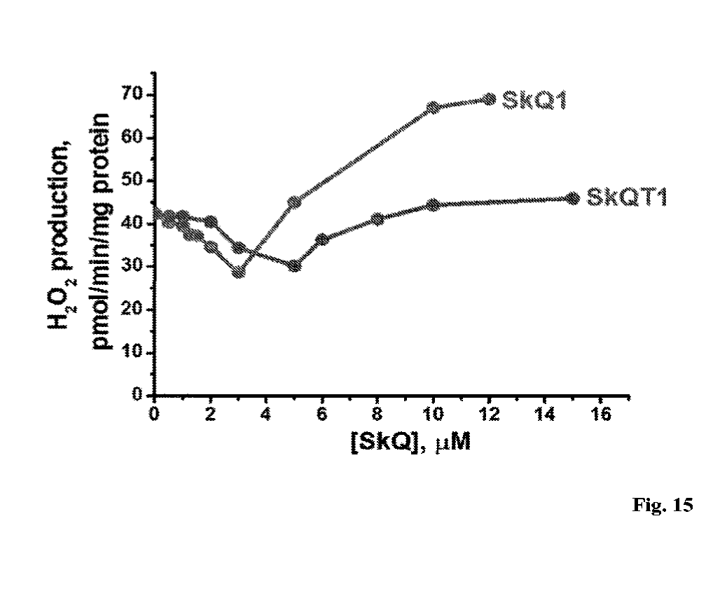

It was demonstrated in previous studies that high concentrations of mitochondrially targeted antioxidants promote peroxide formation by mitochondria (see for example Antonenko et al. 2008). In this experiment we compared such prooxidant effect of SkQ1 and SkQT. Surprisingly SkQT failed to exhibit such prooxidant effect i.e., contrary to SkQ1, the increase of SkQT concentration did not stimulate H₂0₂ formation by rat liver mitochondria up to 15 um concentrations in the medium (see FIG. 15). Incubation medium contained 0.18 M mannitol, 0.07 M sucrose, 0.2 mM Tris-phosphate, 0.5 mM EGTA, pH 7.2, 20 mM Tris-succinate, 2 uM Amplex Red, 5 U horseradish Peroxidase, 6 mM aminotriazole (an inhibitor of catalase) and mitochondria corresponding to 0.25 mg protein. Excitation wavelength was of 563 nm, Emission wavelength was of 585 nm.

### BIOLOGICAL EXAMPLE 7

### Interaction of SkQ Variants with Multi-Drug Resistance Pumps

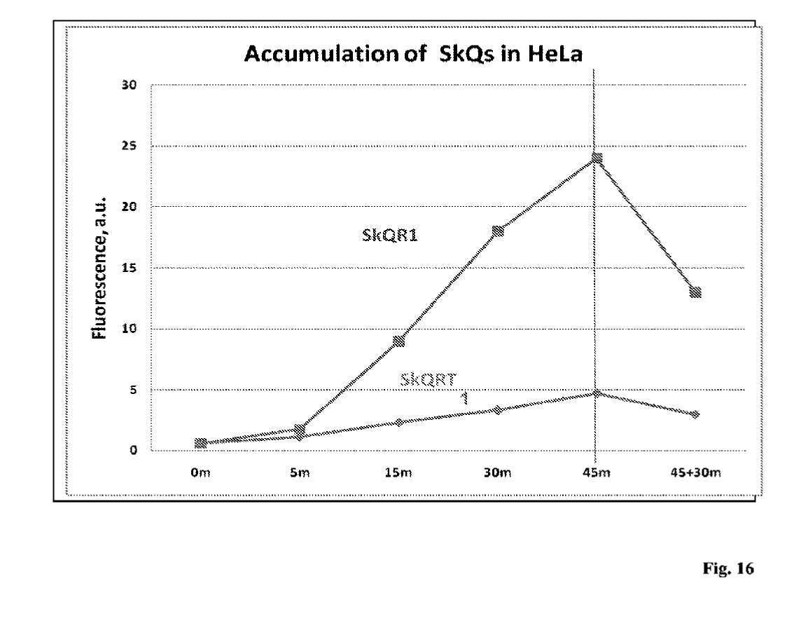

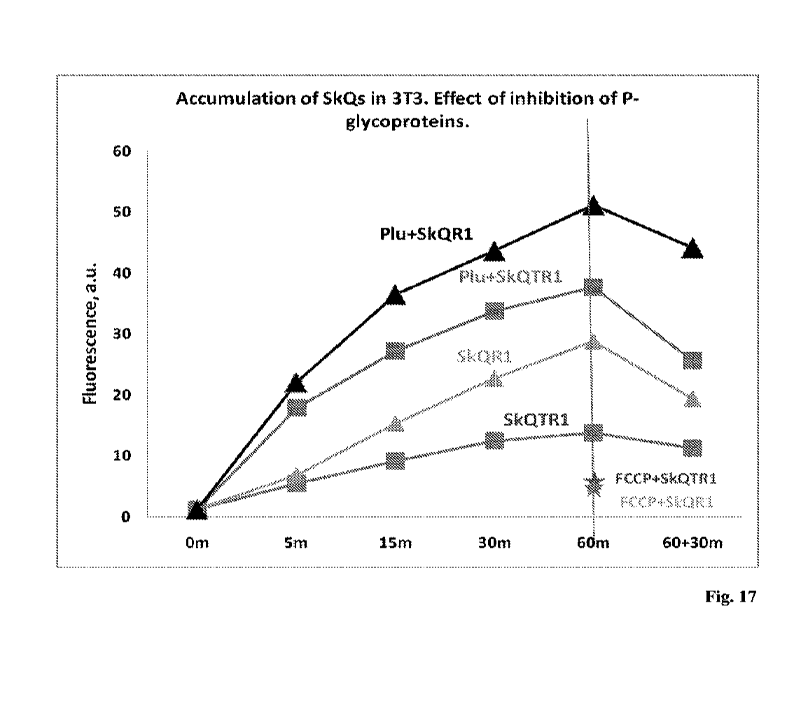

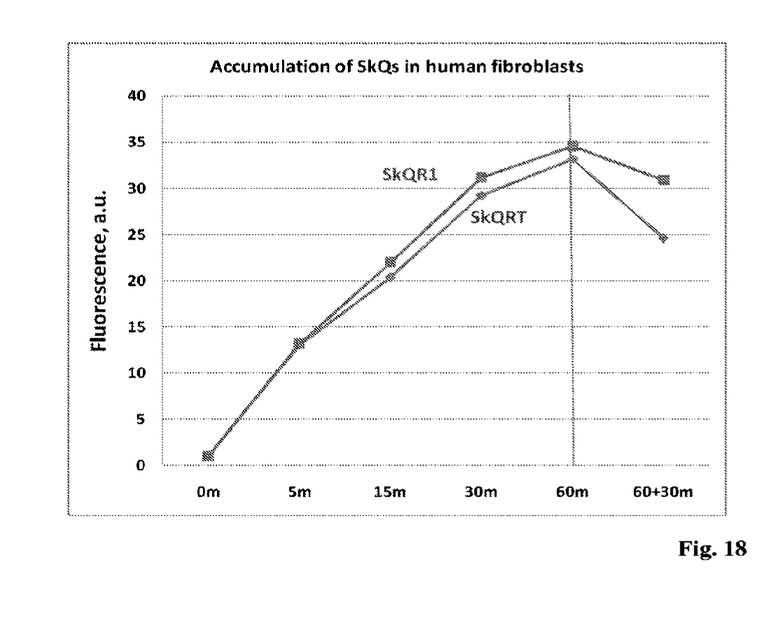

It was reported previously that the fluorescent variant of SkQ-SkQR1 can be recognized and expelled from cells by multi-drug resistance (MDR) proteins (see for example Fetisova et al, 2011, Tsitologiia; 53(6):488-97). In the following experiment we have evaluated MDR expulsion of a timoquinone based SkQRT. HeLa cells were treated with 100 nM SkQRT and the activity of multidrug resistance pumps was estimated by fluorescence measurement using a Beckman Coulter FC 500 flow cytometer. FIG. 16 shows the dynamics of SkQ accumulation in this experiment. Cells were incubated with 100 nM SkQs. After 60 minute, the medium was changed to control medium without SkQs (indicated with vertical line). To prove that the SkQs were expelled from HeLa cells by the MDR system, a corresponding inhibitor—Pluronic® L61 (30 ug/ml) was added to the cells 10 minutes before addition of SkQ variants. The results presented in FIG. 17 show that addition of the MDR inhibitor clearly causes an increased accumulation of both SkQR1 and SkQRT1. It must be noted that MDR pumps are usually activated in tumor cells (for example HeLa), but not in normal non-malignant cells (fibroblasts or 3T3 cells). This is in good correlation with the next experiment where accumulation of SkQR1 and SkQRT1 in primary cultured human fibroblasts (non-tumor cells) was compared. As illustrated in FIG. 18, there was no significant difference in the accumulation of these two compounds.

The results of the experiments described above demonstrate that the efficacy of MDR recognition of SkQ depends on the number of substituted positions in the quinone ring of SkQ.

Recognition by MDR is inversely proportional to the number of substitutions, specifically, the more substitutions within the quinone ring, the less recognition of the SkQ variants by the MDR system. This observation is important in an application of SkQ variants in anti-cancer therapy. One such application would be the treatment of a cancer patient with an SkQ followed by chemotherapy treatment with a pro-oxidant compound. Non-malignant cells (having no MDR system) would accumulate SkQ and thus they will be protected from the pro-oxidant drug and will survive the therapy. Tumor cells will expel most of SkQ via MDR pumps and thus tumor cells will be selectively killed by the chemotherapy. This experimental example suggests the correlation of decreasing MDR-affinity for the following SkQ variants: SkQB>SkQT>SkQ1>SkQ3 (MitoQ); i.e., the MDR system will preferentially recognize SkQT followed by less recognition of SkQ3 and even less recognition of MitoQ. In other words, SkQT and SkQRT are better substrates for MDR pumps than SkQ1 and SkQR1 correspondingly.

### BIOLOGICAL EXAMPLE 8

### SkQB—Mitochondrially Targeted Pro-Oxidant

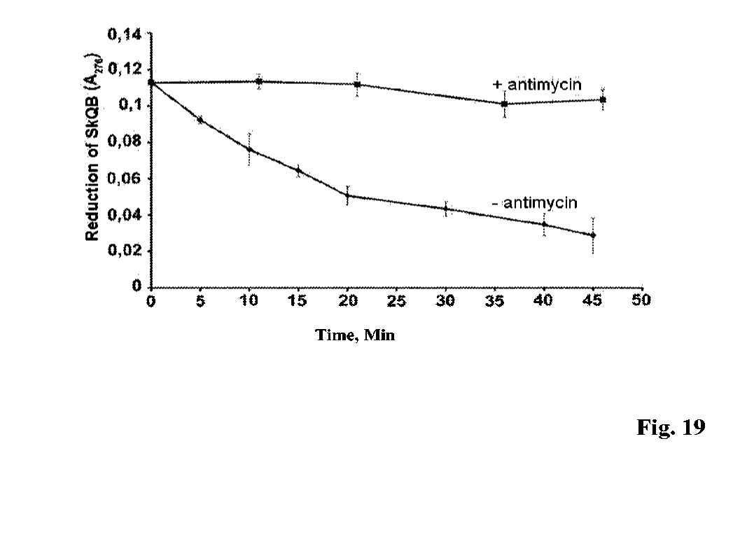

It was unexpectedly found during the study of biological activity of SkQB (10-(1,4-benzoquinonyl-2)decyltriphenylphosphonium), that this variant of mitochondrially targeted quinone SkQ displays very weak antioxidant activity and pronounced pro-oxidant activity. First we have demonstrated that SkQB can be reduced by energized mitochondria in an antimycin-sensitive way (FIG. 19). This was demonstrated using same method as for SkQ1 (Skulachev et al. (2010) Biochim Biophys Acta, 1797(6-7), 878-89). It was shown that kinetics of SkQB reduction is similar to that of SkQ1 (see for comparison Skulachev et al. (2010) Biochim Biophys Acta, 1797(6-7), 878-89).

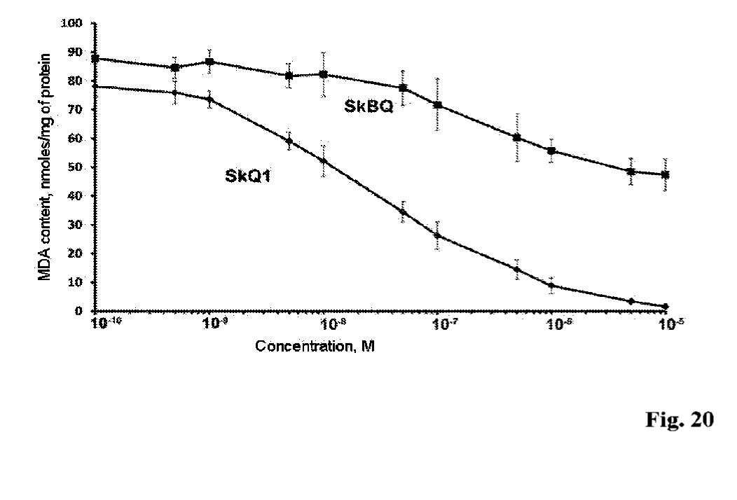

Antioxidant activity was measured in isolated mitochodnria subjected to oxidative damage by the Fenton reaction (method described in Antonenko et al 2008). It was unexpectedly found that SkQB antioxidant activity was lower compared to SkQ1 (FIG. 20).

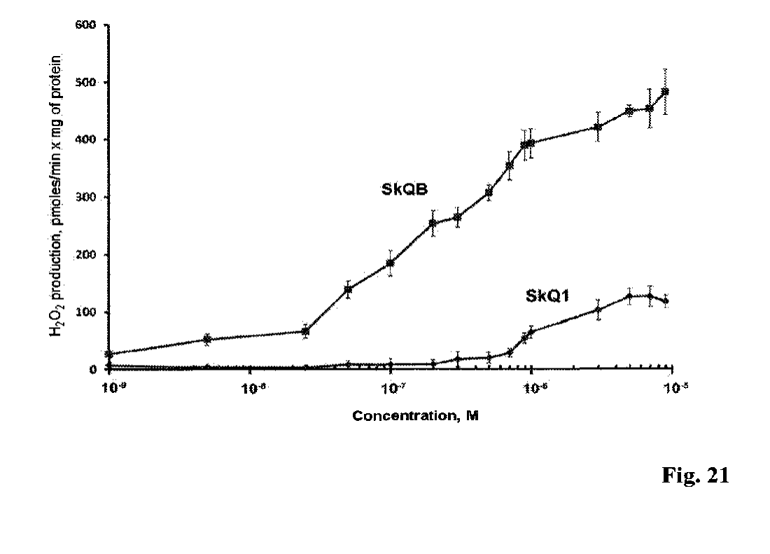

In the next experiment we measured hydrogen peroxide production by mitochondria in the presence of antimycin A (as described in Skulachev et al 2010, Biochim Biophys Acta, 1797(6-7), 878-8). It was found that SkQB stimulates hydrogen peroxide production to a greater extent than SkQ1 (FIG. 21).

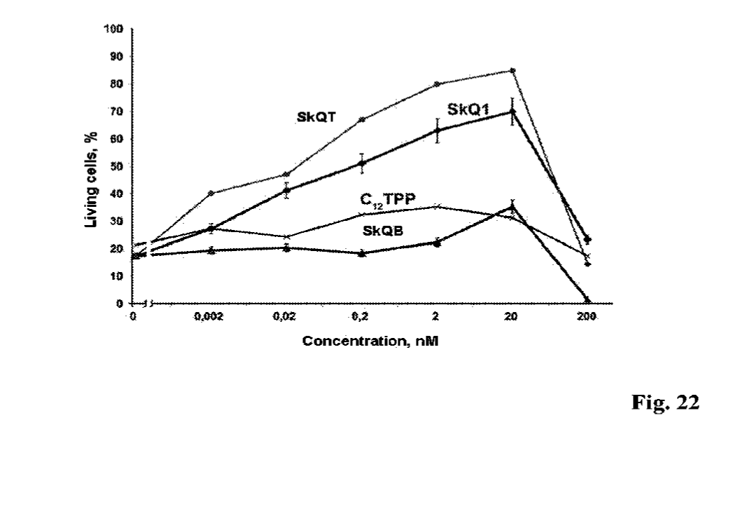

In experiments with human fibroblasts SkQB showed weak protection from H2O2-induced apoptosis in conditions when both SkQ1 and SkQT showed good protection (FIG. 22). These experiments were performed as described in Antonenko et al (2008).

These results demonstrate that the absence of substitutes in the quinone ring of mitochondrially targeted quinine leads to a dramatic decrease of antioxidant activity of the compound and to an increase of pro-oxidant activity. Thus it can be concluded that compounds of the following general formula P:

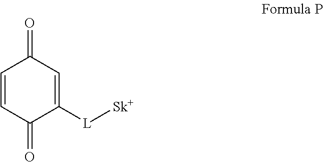

(wherein L is a linker as described herein and Sk⁺ is a lipophyllic cation) can be used as mitochondrially targeted pro-oxidants. SkQB is an example of such mitochondrially targeted pro-oxidant compound.

Some cancers are sensitive to mitochondrial oxidative stress. For example activation of mitochondrial reactive oxygen species production triggers death of prostate cancer cells (see Rico-Bautista E, Zhu W, Kitada S, Ganapathy S, Lau E, Krajewski S, Ramirez J, Bush J A, Yuan Z, Wolf D A. (2013) Oncotarget., 4(8), 1212-29). Thus mitochondrially targeted pro-oxidants can be used as chemotherapeutic agents to treat cancer. As a non-limiting example, SkQB can be used to treat prostate cancer.

1-20. (canceled)

21. A timoquinone-based mitochondrially-targeted antioxidant (MTA) of formula (2): 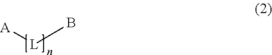
wherein:
A is an timoquinone-derived antioxidant moiety selected from the group consisting of any of Formulas (3-5): 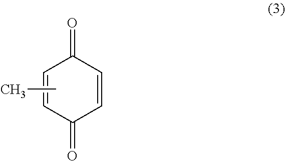
wherein the CH₃ group is attached to any free position of quinone ring; 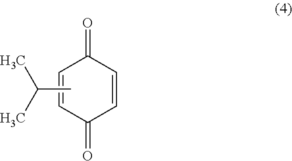
wherein the isopropyl group is attached to any free position of quinone ring; 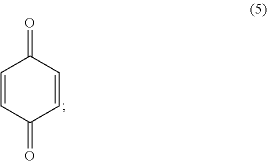
wherein L is a linker group selected from the group consisting of a straight or branched hydrocarbon chain optionally substituted by one or more double or triple bond, or ether bond, or ester bond, or C—S, or S—S, or peptide bond; which is optionally substituted by one or more substituents preferably selected from alkyl, alkoxy, halogen, keto group, amino group; and a natural isoprene chain; wherein n is an integer from 1 to 20; and
wherein B is a targeting group selected from the group consisting of a Skulachev-ion Sk (Sk⁺ Z″) wherein: Sk is a lipophillic cation or a lipophillic metalloporphyrin, and Z is a pharmaceutically acceptable anion; and an amphiphillic zwitterion; and reduced forms thereof.

22. The timoquinone-based MTA according to claim 21 selected from the group consisting of: 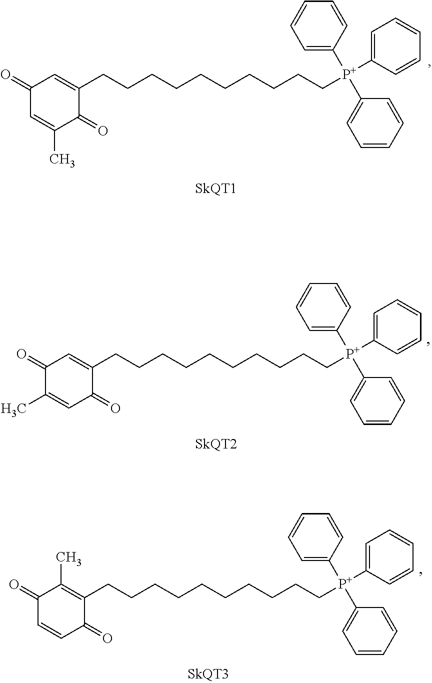 [SMILES: CC1=CC(=O)C=C(CCCCCCCCCC[P+](c2ccccc2)(c2ccccc2)c2ccccc2)C1=O ; CC1=CC(=O)C(CCCCCCCCCC[P+](c2ccccc2)(c2ccccc2)c2ccccc2)=CC1=O ; CC1=C(CCCCCCCCCC[P+](c2ccccc2)(c2ccccc2)c2ccccc2)C(=O)C=CC1=O]
and SkQT which is a mixture of SkQT1, SkQT2 and SkQT3; 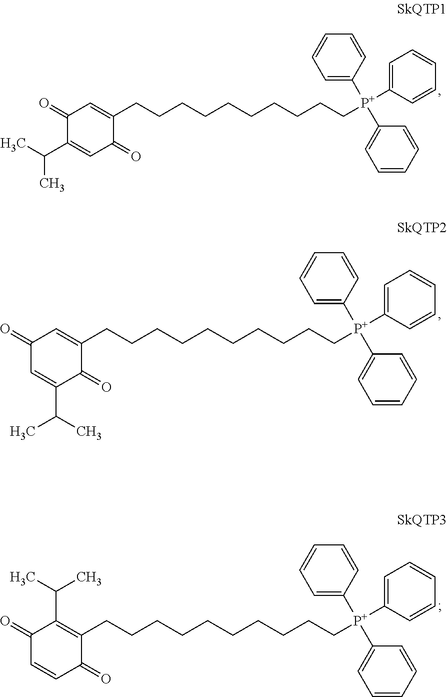 [SMILES: CC(C)C1=CC(=O)C(CCCCCCCCCC[P+](c2ccccc2)(c2ccccc2)c2ccccc2)=CC1=O ; CC(C)C1=CC(=O)C=C(CCCCCCCCCC[P+](c2ccccc2)(c2ccccc2)c2ccccc2)C1=O ; CC(C)C1=C(CCCCCCCCCC[P+](c2ccccc2)(c2ccccc2)c2ccccc2)C(=O)C=CC1=O]
and SkQTP which is a mixture of SkQTP1, SkQTP2 and SkQPT3; 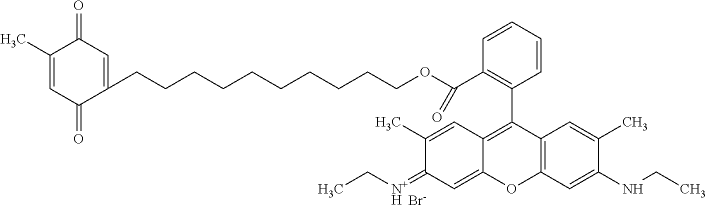
and its isomers SkQRT2, SkQRT3 (analogues for SkQT1-3) and the analogues mixture SkQRT; and 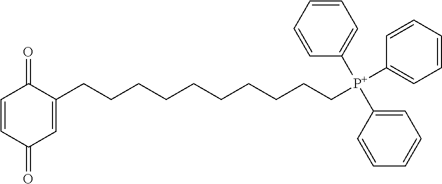 [SMILES: O=C1C=CC(=O)C(CCCCCCCCCC[P+](c2ccccc2)(c2ccccc2)c2ccccc2)=C1]
and reduced forms of any of these compounds.

23. A method for reducing a compound according to claim 21 by an electron-transport chain of mitochondria comprising contacting a cell containing mitochondria with one or more compound according to claim 21.

24. The method according to claim 23, wherein the cell is in the body of a mammal.

25. The method according to claim 24, wherein the mammal is a human.

26. A method for preventing oxidation-induced damage to, or death of, a cell comprising contacting the cell with one or more compound according to claim 21.

27. The method according to claim 26, wherein the cell is in the body of a mammal.

28. The method according to claim 27, wherein the mammal is a human.

29. A method for protecting mitochondria from oxidation-induced damage comprising contacting a cell containing mitochondria with one or more compound according to claim 21.

30. The method according to claim 29, wherein the cell is in the body of a mammal.

31. The method according to claim 30, wherein the mammal is a human.

32. A pharmaceutical formulation comprising one or more compound according to claim 21 and a pharmaceutically acceptable diluent.

33. The mitochondrially targeted pro-oxidant compound SkQB.

34. A method for killing cancer cells comprising contacting the cancer cells with a mitchondrially targeted pro-oxidant compound.

35. The method according to claim 34, wherein the mitchondrially targeted pro-oxidant
compound has the structural formula: 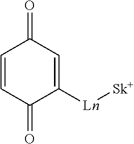
wherein L is a linker group selected from the group consisting of a straight or branched hydrocarbon chain optionally substituted by one or more double or triple bond, or ether bond, or ester bond, or C—S, or S—S, or peptide bond; which is optionally substituted by one or more substituents preferably selected from alkyl, alkoxy, halogen, keto group, amino group; and a natural isoprene chain;
wherein n is an integer from 1 to 20; and
wherein B is a targeting group selected from the group consisting of a Skulachev-ion Sk (Sk⁺ Z″) wherein: Sk is a lipophilic cation or a lipophillic metalloporphyrin, and Z is a pharmaceutically acceptable anion; and an amphiphillic zwitterion.

36. The method according claim 35, wherein the cancer cells are in the body of a mammal.

37. The method according to claim 36, wherein the mammal is a human.

38. The method according to claim 35, wherein the mitochondrially targeted pro-oxidant compound is SkQB.

39. The method according to claim 25, wherein the cancer cells are prostate cancer cells.
